(19) United States
(12) Patent Application Publication
(10) Pub. No.: US 2025/0136715 A1
(43) Pub. Date: May 1, 2025

(54) PHARMACEUTICAL FORMULATION FOR SUBCUTANEOUS ADMINISTRATION OF PROTEINS

(71) Applicants: GCS INSTITUT DE CANCEROLOGIE STRASBOURG EUROPE, STRASBOURG (FR); MEXBRAIN, VILLEURBANNE (FR); UNIVERSITE CLAUDE BERNARD LYON 1, VILLEURBANNE (FR); CENTRE NATIONAL DE LA RECHERCHE SCIENTIFIQUE, PARIS (FR); UNIVERSITE JEAN MONNET SAINT ETIENNE, SAINT ETIENNE (FR); INSTITUT NATIONAL DES SCIENCES APPLIQUEES DE LYON, VILLEURBANNE (FR)

(72) Inventors: Alexandre DETAPPE, STRASBOURG (FR); Xavier PIVOT, TRUCHTERSHEIM (FR); Pierre COLIAT, STRASBOURG (FR); Olivier TILLEMENT, FONTAINES SAINT-MARTIN (FR); Thomas GREA, LYON (FR); François LUX, LYON (FR); Laurent DAVID, LYON (FR)

(73) Assignees: GCS INSTITUT DE CANCEROLOGIE STRASBOURG EUROPE, STRASBOURG (FR); MEXBRAIN, VILLEURBANNE (FR); UNIVERSITE CLAUDE BERNARD LYON 1, VILLEURBANNE (FR); CENTRE NATIONAL DE LA RECHERCHE SCIENTIFIQUE, PARIS (FR); UNIVERSITE JEAN MONNET SAINT ETIENNE, SAINT ETIENNE (FR); INSTITUT NATIONAL DES SCIENCES APPLIQUEES DE LYON, VILLEURBANNE (FR)

(21) Appl. No.: 18/835,795

(22) PCT Filed: Feb. 3, 2023
(86) PCT No.: PCT/EP2023/052732

§ 371 Date: Aug. 5, 2024

(30) Foreign Application Priority Data
Feb. 3, 2022 (EP) 22305120.2

(51) Int. Cl. C07K 16/32; A61K 47/36; C07K 16/28

(52) U.S. Cl. CPC C07K 16/32; A61K 47/36; C07K 16/2887; C07K 16/2896

(57) ABSTRACT

A pharmaceutical formulation of a pharmaceutically active protein including an effective amount of a protein or a combination of proteins, a chitosan A and at least one statistic polysaccharide B. Also, a kit including the at least one first container containing the chitosan A and the statistic polysaccharide B and at least one second container containing the protein or combination of proteins. Further, the pharmaceutical formulation or the kit for use in the treatment of cancer, typically breast or gastric cancer.

### BACKGROUND

The pharmaceutical use of therapeutic protein such as antibodies has increased over the past years. Such therapeutic proteins are mostly administered via the intravenous (IV) route.

Alternative administration pathways include subcutaneous injections.

The advantage of subcutaneous injections is that it allows the medical practitioner to perform it in a rather short intervention with the patient. Moreover, the patient can be trained to perform the subcutaneous injection by himself. Such self-administration is particularly useful during maintenance dosing because no hospital care is needed (reduced medical resource utilization) and could have a positive impact on the patient's quality of life.

Whereas IV delivery can administer large volumes of medication directly into the bloodstream without volume limitation, the injection of parenteral drugs into the hypodermis is generally limited to volumes of less than 2 ml due to the viscoelastic resistance in the subcutaneous (SC) tissue, due to the generated backpressure upon injection, as well as due to the perceptions of pain. The volumes that can be injected in the extracellular matrix of the subcutaneous tissue are thus limited.

For that reason, subcutaneous (SC) formulations of proteins require high protein concentration in the final solution to be injected.

HERCEPTIN™ (Trastuzumab) is a monoclonal antibody directed against the HER2 receptor (anti HER2 antibody) marketed in Europe in the form of a 150 mg lyophilized powder (containing the antibody, α,α-trehalose dihydrate, L-histidine and L-histidine hydrochloride and polysorbate 20) which should be reconstituted for infusions with water for injection to yield an injection dose of approximately 21 mg/ml.

The ROCHE company has developed a subcutaneous formulation of HERCEPTIN® hereinafter referred to as HERCEPTIN® SC. This SC formulation comprises recombinant human hyaluronidase PH20 (rHuPH20), a recombinant human hyaluronidase that induces a local and transient modification of the SC space through degradation of hyaluronan, which is a naturally occurring glycosaminoglycan found throughout the body that creates resistance to bulk fluid flow in the subcutaneous extracellular matrix and limits large-volume SC drug delivery. By degrading the hyaluronan at the local injection site, the hyaluronidase enables SC bulk fluid flow and facilitates the SC delivery of large volumes.

However, high amounts of hyaluronidase may give rise to a risk of inflammation at the injection site. The HERCEPTIN® SC formulation is limited in terms of volume that can be injected precisely because of the presence of this hyaluronidase, which in high quantities can create inflammation. This maximum volume is around 6 mL.

For that reason, the injection of HERCEPTIN® SC formulation must be repeated approximately every two weeks.

Besides, by using circulating antibodies from immunized cynomolgus macaques it has been demonstrated that circulating antibodies specifically recognizing PH-20 bind to the surface of macaque sperm. Therefore, the role of rHuPH20 antibodies on attenuation of fertility in Human cannot be ruled out as anti-sperm antibodies could theoretically interfere with sperm maturation, motility, and in females, anti-sperm antibodies could impede progress through the cervix and uterus, etc. Even if nonclinical studies conducted for SC rHuPH20 containing marketed medicines have not shown effect on reproducibly in several species, reversible infertility has been observed in male and female guinea pigs immunized to produce antibodies to hyaluronidase. The potential risk on reproducibly remains of the major importance, particularly for medicines to be used in young non cancer patients for long duration (multiple sclerosis, inflammatory bowel diseases, etc.).

Another solution for the local administration of antibodies is the prolonged release of antibodies using a suitable support material. Several hydrogels based on synthetic or natural polymers have been proposed for the prolonged release of antibodies. Hydrogels are physical or chemical gels composed of polymer chains swollen by a large amount of water (approximately 70% of the total volume of the hydrogel). For example, hydrogel implants based on poly(ethylene-co-vinyl acetate (EVAc) have been proposed. However, these implants are formed with harsh solvent, which have the potential to degrade the antibodies, and the polymer is not biodegradable, resulting in the patient requiring an additional surgery to remove the implant. An antiangiogenic antibody called bevacizumab (product name Avastin®, Genentech) which targets vascular endothelial growth factor (VEGF) has been examined for various clinical applications, including for the treatment of various cancers, for use in the eye for macular degeneration, and other conditions. One study examined the delivery of bevacizumab from blended glycol chitosan and oxidized alginate hydrogels for possible use in ocular drug delivery, but release was complete after 3 days. Besides, several biopolymers such as alginate-chitosan hydrogels loaded with Bevacizumab and a control IgG. However, these hydrogels were evaluated in vitro only, without studying their biodegradability or biocompatibility. [Flecher et al., Materials Science and Engineering: C, 2016, 59. 806-809].

There is therefore still a need to overcome the drawbacks of the prior art and to provide a pharmaceutical composition comprising a protein or a combination of proteins which may easily be injected by an operator and capable of providing a sustained release of the proteins, while causing little or no inflammation at the injection site.

A low inflammation is advantageous since it may permit to lower the interval of time between two injections, or to select injection areas, which are generally avoided with HERCEPTIN® SC formulation due to the presence of hyaluronidase, such as the abdominal wall. Another objective is to provide a SC formulation of HERCEPTIN® or similar antibodies (such as biosimilars of Herceptin® or other anti-HER2 antibodies) which does not require the presence of hyaluronidase and therefore does not bear the risks of inflammation or toxicity associated with this enzyme.

### SUMMARY

An embodiment E1 of the present disclosure is a pharmaceutical formulation of a pharmaceutically active protein comprising:
an effective amount of a protein or a combination of proteins,
a chitosan A comprising between 90 and 100 mol % of D-glucosamine and between 0 and 15% of N-acetyl-D-glucosamine,
at least one statistic polysaccharide B comprising D-glucosamine, N-acetyl-D-glucosamine and at least one saccharide unit of formula I:

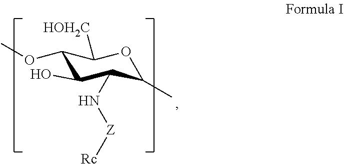

wherein:
   Rc is a hydrophilic group,
   Z is a linker being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur, and
one or more pharmaceutically acceptable excipients.

An embodiment E2 of the present disclosure is a pharmaceutical formulation according to E1, wherein the pharmaceutical formulation forms a hydrogel at physiological pH and osmolarity.

An embodiment E3 of the present disclosure is a pharmaceutical formulation according to E1, wherein Rc is a group having acidic properties, typically comprising chemical functions selected from the group comprising carboxyl group (—COOH), sulfonic group (—SO₂OH), phosphonate groups (—PO(OH)₂), thiol group (—SH), alcohol group (—OH) and groups comprising a chelating agent.

An embodiment E4 of the present disclosure is a pharmaceutical formulation according to E3, wherein Rc is a group comprising a chelating agent, said chelating agent being selected from DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO, and bispidine ([3,7]-diazabicyclo[3.3.1]nonane), preferably selected from DOTAGA, DFO, Bz-DFO, DOTAM, DTPA and bispididine, more preferably Rc is DOTAGA.

An embodiment E5 of the present disclosure is a pharmaceutical composition according to E1, wherein said statistic polysaccharide B is of the general formula II:

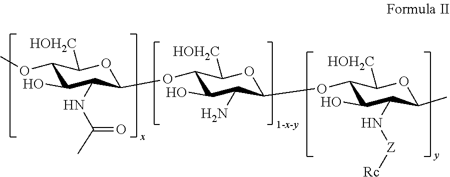

wherein:
   Rc is a hydrophilic group,
   Z is a linker being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur,
   x is comprised between 0.01 and 0.5,
   y is comprised between 0.05 and 0.5,
   a ratio y/x is greater than 0.2, preferably greater than 1,
   a sum x+y is greater than 0.1.

An embodiment E6 of the present disclosure is a pharmaceutical formulation according to E5, wherein Rc is a group having acidic properties, typically selected from the group comprising carboxyl group (—COOH), sulfonic group (—SO2OH), phosphonate groups (—PO(OH)₂), thiol group (—SH), alcohol group (—OH) and groups comprising a chelating agent.

An embodiment E7 of the present disclosure is a formulation according to E6, wherein Rc is a group comprising a chelating agent, said chelating agent being selected from DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO, and bispidine ([3,7]-diazabicyclo[3.3.1]nonane), preferably selected from DOTAGA, DFO, Bz-DFO, DOTAM DTPA, and bispidine, more preferably Rc is DOTAGA.

An embodiment E8 of the present disclosure is a pharmaceutical formulation according to E1, wherein said statistic polysaccharide B is of the general formula III:

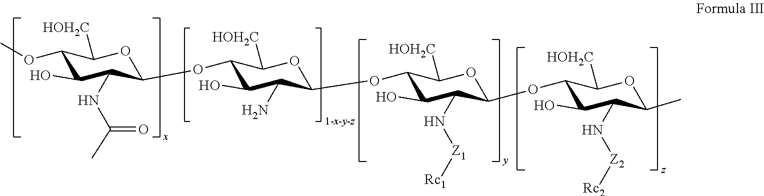

Wherein:
   R_{c1} and R_{c2} are not identical, and are hydrophilic group,
   Z1 and Z2, identical or not, are linkers being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur,
   x is comprised between 0.01 and 0.5, preferably between 0.01 and 0.1 and more preferably between 0.05 and 0.1,
   y is comprised between 0.01 and 0.5, preferably between 0.05 and 2
   z is comprised between 0 and 0.2,
   a ratio y/x is greater than 0.2, preferably greater than 1,
   a sum x+y is greater than 0.1.

An embodiment E9 of the present disclosure is a pharmaceutical formulation according to E8, wherein Rc1 and Rc2 are a group having acidic properties, typically selected from the group comprising carboxyl group (—COOH), sulfonic group (—SO₂OH), phosphonate groups (—PO(OH)₂), thiol group (—SH), alcohol group (—OH) and groups comprising a chelating agent.

An embodiment E10 of the present disclosure is a pharmaceutical formulation according to E9, wherein Rc1 and/or Rc2 is/are a chelating agent, Rc1 and/or Rc2 are preferably selected from DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO and bispidine ([3,7]-diazabicyclo[3.3.1]nonane), preferably selected from DOTAGA, DFO, Bz-DFO, DOTAM, DTPA and bispidine, more preferably Rc1 is DOTAGA and Rc2 is DFO or Bz-DFO.

An embodiment E11 of the present disclosure is a pharmaceutical formulation according to anyone of preceding embodiments, wherein Z, Z1 and Z2 are independently selected from the group consisting of a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur, an alkyl chain comprising between 1 and 12 carbon atoms, said chain being linear or branched, and an alkenyl chain comprising between 2 and 12 carbon atoms, said chain being linear or branched, said alkyl and alkenyl chains could be interrupted by one or more aryl groups in C₆-C₁₀, and/or one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR′ said alkyl and alkenyl chains could be substituted by one or more of the atoms or groups selected from the group consisting of —OR′, —COOR′, —SR′, —NR′2, each R′ being independently H or an alkyl in C₁-C₆, preferably each of Z, Z1 and Z2 is independently selected from the group consisting of a single bond, an alkyl chain comprising between 1 and 12 carbon atoms, said chain being linear or branched, said alkyl chain could be interrupted by one or more aryl groups in C6-C10, and/or one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′ each R′ being independently H or an alkyl in C₁-C₆.

An embodiment E12 of the present disclosure is a pharmaceutical formulation according to E11 wherein, each of Z, Z1 and Z2 is an alkyl chain comprising between 1 and 12 carbon atoms.

An embodiment E13 of the present disclosure is a pharmaceutical formulation according to E11 wherein, each of Z, Z1 and Z2 is a polyethylene glycol (PEG) fraction.

An embodiment E14 of the present disclosure is a pharmaceutical formulation according to any one of E1 to E13, wherein the statistic polysaccharide B has a mean molecular weight between 100 kDa and 1000 kDa, more advantageously between 200 kDa and 750 kDa, even more advantageously between 250 kDa and 500 kDa and most advantageously between 300 kDa et 400 kDa.

An embodiment E15 of the present disclosure is a pharmaceutical formulation according to any one of E1 to E14 for subcutaneous administration.

An embodiment E16 of the present disclosure is a pharmaceutical formulation according to any one of E1 to E15, wherein the hydrogel releases the protein over a period between 10 and 100 days, preferably between 20 and 65 days from the day of administration.

An embodiment E17 of the present disclosure is a pharmaceutical formulation according to any one of E1 to E16, wherein the viscosity of the formulation is comprised between 10 and 500 Pa·s, as measured at room temperature by rotational rheometry in plane cone geometry at a shear rate of between 0.01 and 0.001 s⁻¹, for example of 0.001 s⁻¹ or 0.01 s⁻¹, in particular of 0.01 s⁻¹.

An embodiment E18 of the present disclosure is a pharmaceutical formulation according to any one of E1 to E17, wherein the pH of the formulation is comprised between 5.0 and 6.5, preferably between 5.5 and 6.0.

An embodiment E19 of the present disclosure is a pharmaceutical formulation according to any one of E1 to E18, wherein the osmolarity of the formulation is comprised between 50 and 600 mOsm/L, preferably comprised between 100 and 600 mOsm/L, more preferably between 250 and 450 mOsm/L, or between 50 and 300 mOsm/L, preferably between 50 and 250 mOsm/L.

An embodiment E20 of the present disclosure is a pharmaceutical formulation according to any one of E1 to E19, comprising:
between 5 and 200 g/l, preferably between 10 and 200 g/l, more preferably between 10 and 150 g/l of chitosan A, and between 10 and 200 g/l, preferably between 20 and 100 g/l of statistic polysaccharide B, and
between 1 g/l and 200 g/l, preferably between 50 et 100 g/l of protein or combination of proteins.

An embodiment E21 of the present disclosure is a pharmaceutical formulation according to any of E1 to E20, wherein the protein has a molecular weight comprised between 10 kDa and 250 kDa, preferably between 20 kDa and 250 kDa, preferably between 100 kDa and 250 kDa, preferably between 120 kDa and 250 kDa, preferably between 150 kDa and 250 kDa.

An embodiment E22 of the present disclosure is a pharmaceutical formulation according to any one of embodiments E1 to E21, wherein the protein or combination of proteins is selected from the group consisting of an antibody, an enzyme, a fusion protein, and a combination thereof.

An embodiment E23 of the present disclosure is a pharmaceutical formulation according to any one of E1 to E22, wherein the formulation is free of hyaluronidase enzyme.

An embodiment E24 of the present disclosure is a pharmaceutical formulation according to any one of E1 to E23, wherein the protein or combination of proteins includes an antibody or an antigen-binding fragment of an antibody.

An embodiment E25 of the present disclosure is a pharmaceutical formulation according to E24, wherein said antibodies or combination of antibodies is selected from the group consisting of (i) a chimeric, human or humanized antibody, and (ii) an antibody-drug conjugate.

An embodiment E26 of the present disclosure is a pharmaceutical formulation according to any one of E1 to E25, wherein the protein or combination of proteins is an antibody or combination of antibodies which binds an antigen selected from the group of HER2 and CD20.

An embodiment E27 of the present disclosure is a pharmaceutical formulation according to E27, wherein the antibody is selected from the group consisting of trastuzumab, rituximab and pertuzumab or combinations thereof.

An embodiment E28 of the present disclosure is a pharmaceutical formulation according to any one of E27 or E28, wherein the formulation comprises a combination of trastuzumab and pertuzumab.

An embodiment E29 of the present disclosure is a pharmaceutical formulation according to any one of E1 to E28, further comprising at least one pharmaceutically acceptable excipient selected from solvents, stabilizers, surfactants, buffering agents, antimicrobial preservatives, protectants, antioxidants, chelating agents, and bulking agents.

An embodiment E30 of the present disclosure is a pharmaceutical formulation according to any one of E1 to E30, wherein the formulation comprises:
between 1 g/l and 200 g/l, preferably between 50 et 100 g/l of an antibody or a combination of antibodies, in particular in particular trastuzumab, rituximab, pertuzumab, or daratumumab, or a combination of trastuzumab and pertuzumab,
between 5 and 200 g/l, preferably between 10 and 200 g/l, more preferably between 10 and 150 g/l of chitosan A and
between 20 and 400 g/l, preferably between 20 and 300 g/l of statistic polysaccharide B, and
at least one excipient selected from:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

An embodiment E31 of the present disclosure is a kit characterized in that it comprises:
at least one first container containing the chitosan A and the statistic polysaccharide B as defined in any one of E1 to E12,
at least one second container containing the protein or combination of proteins as defined in any one of E22 to E29.

An embodiment E32 of the present disclosure is a kit according to E31 for preparing a ready-to-use injectable formulation according to the present disclosure.

An embodiment E33 of the present disclosure is a pharmaceutical formulation according to any one of claims E1 to E31 or a kit according to any one of E32 or E33, for use in the treatment of cancer, typically breast or gastric cancer, or of nonmalignant diseases.

An embodiment E34 of the present disclosure is a method of treating a cancer in a subject in need thereof, said method comprising subcutaneously administering a therapeutically effective amount of an antibody or a combination of antibodies which bind an antigen selected from the group of HER2 and CD20 according to any one of E27 to E29 in said subject.

### LEGEND OF THE FIGURES

FIG. 1 illustrates a gel formed in Hemosol B0 from a MEX-CD2-I-tmb solution FIG. 2 illustrates the in situ gelation of a MEX-CD2-I-tmb solution with fluorescent marking the solution (FIG. 2A), and the gel obtained after complete gelation in physiological serum (FIG. 2B).

FIG. 3 illustrates the stability ranges of MEX-CD2-I solutions as a function of pH and osmolarity.

FIG. 4 illustrates the time tracking of mouse weight after subcutaneous injection of 200 μL of MEX-CD2-I-tmb solutions loaded with 100 g/L of trastuzumab antibody. N=9 mice.

FIG. 5 illustrates the hematoxylin and eosin (H&E) labelling at day 20 after subcutaneous injection of 200 μL of a MEX-CD2-I-tmb solution (corresponding to a dose of 15 mg of trastuzumab).

FIG. 6 illustrates the time course of the presence in plasma of trastuzumab antibodies after subcutaneous injection of 200 μL of a MEX-CD2-I-tmb solution (corresponding to a dose of 15 mg of trastuzumab) vs. the subcutaneously injection of a Roche SC HERCEPTIN® formulation (corresponding to a dose of 15 mg of trastuzumab).

FIG. 7 illustrates the monitoring of the presence of a solution MEX-CD2-I with fluorescent marking after injection.

FIG. 8 illustrates the longitudinal monitoring of the antibody-loaded hydrogel degradation by fluorescence imaging.

FIG. 9 shows the percentage of fluorescence compared to the initial fluorescence of the hydrogel over time.

FIG. 10 illustrates the percentage of gadolinium present in each organs/regions compared to the maximum amount of gadolinium complexed in the gel initially for solution with and without trastuzumab.

FIG. 11 illustrates (A) the T₁ MRI signal of the hydrogel-loaded trastuzumab implanted in the neck (200 μL) of the mice (n=3)—Dashed lines represent the hydrogel contour—and (B) the volume curves of the hydrogels upon different volumes and ratio of trastuzumab loaded.

FIG. 12 shows a comparison of the volume injected of hydrogel-loaded trastuzumab implanted subcutaneously. 50 μL and 200 μL of hydrogel-loaded trastuzumab (at 100 G/L trastuzumab) implanted subcutaneously (n=3) provide the same degradation profile.

FIG. 13 shows the immunofluorescence staining of the pro-inflammatory neutrophils localized at the injection site at day 5 post-implantation of the hydrogel-loaded trastuzumab.

FIG. 14 shows the cumulative release of monoclonal antibodies (trastuzumab or rituximab) and combination of monoclonal antibodies (trastuzumab and pertuzumab) in PBS at 37° C.

FIG. 15 shows the results regarding the cumulative release of fragments of monoclonal antibodies (Fab′2 and VHH) in PBS at 37° C.

FIG. 16 shows the SEM imaging of a gel formed in a PBS solution from a polymeric solution with and without trastuzumab for different magnification rates.

### DETAILED DESCRIPTION

A first aspect of the disclosure is a pharmaceutical formulation comprising:
a therapeutically effective amount of a protein or a combination of proteins,
a chitosan A comprising between 90 and 100 mol % of D-glucosamine and between 0 and 15% of N-acetyl-D-glucosamine,
at least one statistic polysaccharide B comprising D-glucosamine, N-acetyl-D-glucosamine and at least one saccharide unit of formula I:

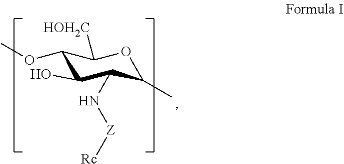

wherein:
   Rc is a hydrophilic group,
   Z is a linker being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur, and
one or more pharmaceutically acceptable excipients.

### Polysaccharides

According to the present disclosure, “chitosan” means a natural polymer of polysaccharide type of D-glucosamine (GlcN) or co-polysaccharide type, consisting of a random distribution (statistic copolysaccharide) or not (blocks copolysaccharide) of D-glucosamine and N-acetyl-D-glucosamine (GlcNAc), linked by glycosidic bonds of β(1->4) type.

According to the present disclosure, the mean molecular weight Mw of polysaccharides such as Chitosan A and polysaccharide B are measured by steric exclusion chromatography, the method being described in “Physico-chemical studies of the gelation of chitosan in a hydroalcoholic medium” A. MONTEMBAULT, C. VITON, A. DOMARD, Biomaterials, 26(8), 933-943, 2005.

The proportion of N-acetyl-D-glucosamine is calculated by using H¹NMR, following the Hirai's methodology (A. HIRAI, H ODANI, A. NAKAJIMA, Polymer Bulletin, 26 (1), 87-94, 1991).

According to the present disclosure, crystallinity ratio represents the proportion of crystalline material. Regarding polysaccharides, the crystallinity ratio is often determined using X diffraction (Alexander, L. E., ‘X-ray Diffraction Methods in Polymer Science’, Wiley-Interscience, New York, 1969, p. 137).

### Chitosan A

The pharmaceutical formulation of the disclosure comprises a chitosan A. The formulation may have:
a crystallinity ratio on chitosan A of at least 10% by weight relative to the total dry mass of the formulation, and
a crystallite size of chitosan A of less than 20 nm.

The crystallinity ratio on chitosan A could be between 10 and 25%. The crystallites of chitosan A act as physical cross-linking junctions which are mandatory for constituting a gel. The size of the crystallites may be, for example between 1 and 20 nm.

Advantageously, chitosan A has a mean molecular weight Mw between 100 kg/mol and 1000 kg/mol, preferably between 200 kg/mol and 700 kg/mol.

According to a preferred embodiment, chitosan A has between 1 mol % and 9 mol % of N-acetyl-D-glucosamine, preferably between 2 mol % and 8 mol %, more preferably between 3 mol % and 7 mol %, even more preferably between 4 mol % and 6 mol % and most preferably 5 mol %.

### Statistic Polysaccharide B

The formulation comprises a statistic polysaccharide B.

Advantageously, the statistic polysaccharide B has a mean molecular weight between 100 kDa and 1000 kDa, more advantageously between 200 kDa and 750 kDa, even more advantageously between 250 kDa and 500 kDa and most advantageously between 300 kDa and 400 kDa.

Preferably, Rc is a group having acidic properties, typically selected from the group comprising carboxyl group (—COOH), sulfonic group (—SO₂OH), phosphonate groups (—PO(OH)₂), thiol group (—SH), alcohol group (—OH) and groups comprising a chelating agent.

In specific embodiments where Rc is a chelating agent, Rc may be selected from DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO and bispidine ([3,7]-diazabicyclo[3.3.1]nonane), preferably selected from DOTAGA, DFO, Bz-DFO, DOTAM, DTPA and bispidine, more preferably Rc is DOTAGA.

The chelating agents of polysaccharide B preferably participate in the gelation of the polysaccharide mixture and give the hydrogel swelling properties. Besides, the chelating agent preferably promotes the formation of a hydrogel containing non-crystallized areas, which may result in a good biodegradability of the hydrogel.

Chitosan A promotes the formation of crystallites responsible for the gel state of the materials in contact with body fluids.

According to the present disclosure, the expression “polysaccharide mixture” refers to Chitosan A and polysaccharide B.

In an embodiment, the statistic polysaccharide B is of the following general formula II:

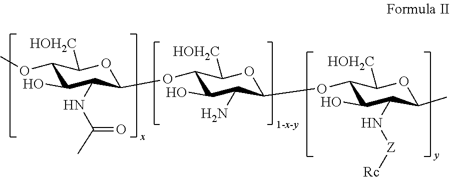

wherein:
Rc is a hydrophilic group,
Z is a linker being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur,
x is comprised between 0.01 and 0.5,
y is comprised between 0.05 and 0.5,
a ratio y/x is greater than 0.2, preferably greater than 1,
a sum x+y is greater than 0.1.

In some embodiments, the statistic polysaccharide B comprises two different units of formula I. In these embodiments; the statistic polysaccharide B is of the following general formula III:

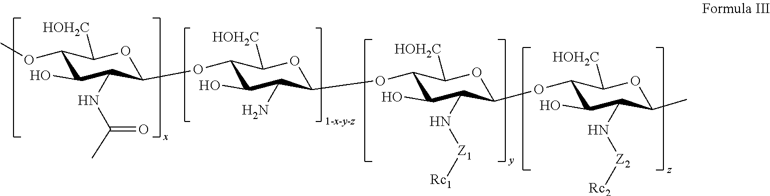

### Wherein:

Rc₁ and Rc₂ are not identical, and are hydrophilic group,
Z1 and Z2, identical or not, are linkers being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur,
x is comprised between 0.01 and 0.5, preferably between 0.01 and 0.1 and more preferably between 0.05 and 0.1,
y is comprised between 0.01 and 0.5, preferably between 0.05 and 2
z is comprised between 0 and 0.2,
a ratio y/x is greater than 0.2, preferably greater than 1,
a sum x+y is greater than 0.1.

Preferably, Rc₁ and Rc₂ are a group having acidic properties, typically selected from the group comprising carboxyl group (—COOH), sulfonic group (—SO2OH), phosphonate groups (—PO(OH)₂), thiol group (—SH), alcohol group (—OH) and groups comprising a chelating agent.

In embodiments where Rc and/or Rc2 is/are a chelating agent, Rc and/or Rc2 are preferably selected from DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO and bispidine ([3,7]-diazabicyclo[3.3.1]nonane), preferably selected from DOTAGA, DFO, Bz-DFO, DOTAM, DTPA and bispidine, more preferably Rc1 is DOTAGA and Rc2 is DFO or Bz-DFO.

A precursor of Bz-DFO chelating group is p-NCS-Bz-DFO (Cas No: 1222468-90-7) whose NCS group gives a thiourea when it reacts on an amine, and is therefore no longer NCS once grafted onto a molecule such as polysaccharide B.

Rc, Rc₁ and Rc₂ being Chelating Groups

As mentioned above, Rc groups in formula I and in formula II as well as Rc₁ and Rc₂ groups in formula III may be chelating groups. In embodiments Rc, Rc₁ and RC₂ are chelating groups, Rc, Rc₁ and Rc₂ can chelate one or more metals by forming a complex.

In an embodiment, less than 10% of the chelating groups, preferably less than 5% are chelated with a cation, in particular a metallic cation. In case the chelating groups are in a free form a good capture of metals and polysaccharide B has also a strong hydrophilic behaviour what implies good swelling properties.

Each of Rc, Rc₁ and Rc₂ can comprise one or more coordination sites. Preferably, coordination sites are a nitrogen or an oxygen atom. Advantageously, each of Rc, Rc₁ and Rc₂ comprises between 4 and 8 coordination sites, more advantageously between 6 and 8 coordination sites and even more advantageously each of Rc, Rc₁ and Rc₂ comprises 8 coordination sites.

In the present disclosure, a coordination site means a unique function able to chelate a metal. For example, an amine function represents one coordination site by forming a dative bond between the nitrogen of the amine and a metal an a hydroxamic acid function also represents one coordination site by forming a dative bond between the oxygen of the carbonyl moiety and a metal and by a covalent bond between the oxygen of the N-oxyde moiety and the very same metal, the coordination site forming a 5 links ring.

Z, Z₁ and Z₂

As mentioned above, Z, Z₁ and Z₂, are linkers being a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur.

Choice of Z, Z₁ and Z₂ depends essentially on Rc, Rc₁ and Rc₂ and of the metal eventually chelated. Indeed, notably for steric reasons, Rc, Rc₁ and Rc₂ could be more or less close to the 6 links ring.

In some embodiments, Z, Z₁ and Z₂ are independently selected from the group consisting of a single bond, a hydrocarbonated chain comprising between 1 and 12 carbon atoms, said chain being linear or branched, saturated or unsaturated and optionally comprising one or more heteroatoms preferably selected from nitrogen, oxygen and sulfur, an alkyl chain comprising between 1 and 12 carbon atoms, said chain being linear or branched, and an alkenyl chain comprising between 2 and 12 carbon atoms, said chain being linear or branched, said alkyl and alkenyl chains could be interrupted by one or more aryl groups in C₆-C₁₀, and/or one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR′ said alkyl and alkenyl chains could be substituted by one or more of the atoms or groups selected from the group consisting of —OR′, —COOR′, —SR′, —NR′₂, each R′ being independently H or an alkyl in C₁-C₆.

Advantageously, each of Z, Z₁ and Z₂ is independently selected from the group consisting of a single bond, an alkyl chain comprising between 1 and 12 carbon atoms, said chain being linear or branched, said alkyl chain could be interrupted by one or more aryl groups in C₆-C₁₀, and/or one or more heteroatoms or groups selected from the group consisting of —O—, —S—, —C(O)—, —NR′—, —C(O)NR′—, —NR′—C(O)—, —C(S)NR′—, —NR′—C(S)—, —NR′—C(S)—NR′ each R′ being independently H or an alkyl in C₁-C₆.

In a particular embodiment, each of Z, Z₁ and Z₂ is an alkyl chain comprising between 1 and 12 carbon atoms.

In another particular embodiment, each of Z, Z₁ and Z₂ is a polyethylene glycol (PEG) fraction.

### Process for Preparing Polysaccharide B

Polysaccharide B could be obtained with a process comprising the three following successive steps:
Step 1: solubilizing a chitosan in an acidic solution at a pH between 4 and 5;
Step 2: partial acetylation of amine functions of the chitosan solubilized in step 1;
Step 3: functionalization of at least a part of the remaining amine functions after step 2.

Step 3 could be subdivided in several under steps, notably when Z, Z₁ and/or Z₂ is/are a hydrocarbonated chain as defined above.

In embodiments where Z, Z₁ and/or Z₂ is/are a hydrocarbonated chain as defined above, step 3 may comprise an under step 3-1 consisting in grafting said hydrocarbonated chain on at least a part of the remaining amine functions after step 2, and then an under step 3-2 consisting in grafting of Rc, Rc₁ and/or Rc₂. Alternatively, step 3 does not comprise an under step. In this alternative, said hydrocarbonated chain is coupled with Rc, Rc₁ and Rc₂, said step 3 is, in this case, performed with a molecule comprising Rc, Rc₁ or and said hydrocarbonated chain.

In an alternative manner, polysaccharide B could be obtained starting from a chitosan having the suitable number of N-acetyl-D-glucosamine units. In this alternative, the above step 2 has not to be performed, thus, in this alternative the process for obtaining polysaccharide B comprises at least the two following successive steps:
Step 1b: solubilizing a chitosan, comprising N-acetyl-D-glucosamine units, in an acidic solution at a pH between 4 and 5;
Step 2b: functionalization of at least a part of the amine functions of said chitosan, comprising N-acetyl-D-glucosamine units solubilized at step 1.

In the same manner as above for said step 3, said step 2b could be subdivided in several under steps, notably when Z, Z₁ and/or Z₂ is/are a hydrocarbonated chain as defined above.

In embodiments where Z, Z₁ and/or Z₂ is/are a hydrocarbonated chain as defined above, step 2b may comprise an under step 2b-1 consisting in grafting said hydrocarbonated chain on at least a part of the amine functions, and then an under step 2b-2 consisting in grafting of Rc, Rc₁ and/or Rc₂ on said hydrocarbonated chain. Alternatively, step 2b does not comprise an under step. In this alternative, said hydrocarbonated chain is coupled with Rc, Rc₁ and Rc₂, said step 2b is, in this case, performed with a molecule comprising Rc, Rc₁ or and said hydrocarbonated chain.

### Contents of Polysaccharides

The content of chitosan A in the pharmaceutical formulation of the disclosure is preferably comprised between 5 and 200 g/l, preferably between 10 and 200 g/l, more preferably between 10 and 150 g/l of chitosan A.

The content of statistic polysaccharide B in the pharmaceutical formulation of the disclosure is preferably comprised between 10 and 200 g/l, preferably between 20 and 100 g/l of statistic polysaccharide B.

The chitosan A/statistic polysaccharide B weight ratio preferably ranges between 5/1 and 1/5, preferably between 1/1 and 1/5, preferably between 1/1 and 1/3.

In an embodiment, the chitosan A/statistic polysaccharide B weight ratio is about 1/2, preferably is 1/2.

In an embodiment, the pharmaceutical composition comprises between 2% and 10%, preferably comprises between 2%, 3%; 4%, 5%, 6%, 7%, or 8% and 10%, more preferably comprises between 3% and 8% of chitosan A and statistic polysaccharide B, % expressed in weight relative to the weight of the pharmaceutical composition.

### Viscosity

The pharmaceutical formulation of the disclosure can easily be injected in a tissue due to its rheological properties while being able to forms, in vivo, a biodegradable hydrogel that entraps the proteins and enables their controlled and prolonged release in the tissue.

The pharmaceutical formulation of the disclosure is preferably under the form of a liquid formulation, more preferably of a liquid injectable formulation.

According to the present disclosure, a “liquid injectable formulation” means a liquid formulation having viscosity sufficiently low to allow a good injectability trough a gauge needle.

The term “injectability” or “syringeability,” as generally used herein, refers to the injection performance of the pharmaceutical formulation of the disclosure through a syringe equipped with a 18-32 gauge needle, preferably a 20-27 gauge needle.

The pharmaceutical formulation of the disclosure preferably has a viscosity comprised between 10 and 500 Pa·s, preferably between 10 and 300 Pa·s., more preferably between 15 and 250 Pa·s, as measured at room temperature by rotational rheometry in plane cone geometry at a shear rate of between 0.01 and 0.001 s⁻¹, for example of 0.001 s⁻¹ or 0.01 s⁻¹, preferably of 0.01 s⁻¹.

In an embodiment, the viscosity of the pharmaceutical formulation is comprised between 10 and 80 Pa·s, preferably between 10 and 50 Pa·s, more preferably between 10 and 30 Pa·s, or a viscosity comprises between 80 and 250 Pa·s, preferably between 80 and 120 Pa·s, for example of about 100 Pa·s.

The term “viscosity” refers to the resistance of a substance (typically a liquid) to flow. Viscosity is related to the concept of shear force; it can be understood as the effect of different layers of the fluid exerting shearing force on each other, or on other surfaces, as they move against each other. The units of viscosity are Ns/m², known as Pascal-seconds (Pa·s).

The viscosity of the pharmaceutical formulation of the disclosure is typically measured by rotational rheometry in plane cone geometry using a viscometer with imposed deformation, such as an Advanced Rheometer AR2000 from TA instruments. Briefly, the fluid is subjected to shear between two surfaces, one fixed and one rotating around its axis. In practice, these two surfaces are commonly a cone and a plane. The shear gradient is determined by the geometry of the surface on the one hand, and the speed of rotation on the other hand. The shear stress is calculated from the measurement of the torque transmitted by the sample to be characterized. A detailed description of this method can be found in El Kissi et al, Rheology, 10, 13-39 (2006). In the present invention, measurements are performed at room temperature (e.g. about 20° C., preferably about 25° C.) and are typically made using a 4° and 25 mm cone plane geometry at a continuous shear rate of between 0.01 and 0.001 s⁻¹, for example of 0.01 s⁻¹ or 0.001 s⁻¹, in particular of 0.01 s⁻¹ (viscosity measurement method (i)). Alternatively, measurements may be performed at room temperature (e.g. about 20° C., preferably about 25° C.) using a C35/2° Ti L cone plate geometry with a flow sweep linear study conducted by scanning shear rate from 10⁻² to 10³ s⁻¹ (viscosity measurement method (ii)). Viscosity measurement method (i) is preferably performed using an Advanced Rheometer AR2000 from TA instruments with a 4° and 25 mm planar cone geometry. Viscosity measurement method (ii) is preferably performed using a RheoStress 600 rheometer from Thermo Scientific HAAKE using a C35/2° Ti L cone plate geometry. In viscosity measurement methods (i), the viscosity values may be measured at relatively low shear rates. However, it can be noted that for low viscosity solutions, e.g. for solutions having a viscosity lower than 20 Pa·s, the viscosity values measured according to viscosity measurement method (i) at shear rate values of about 0.001 s⁻¹ can in some cases be distorted because it corresponds to the measurement limit of the machine. For such low viscosity solutions, viscosity measurement is preferably carried out according to viscosity method (i) at shear rates higher than 0.001 s⁻¹, typically at shear rates of about of 0.01 s⁻¹, preferably at a shear rate of 0.01 s⁻¹, or according to viscosity measurement method (ii).

Prior to the measurement, the zero deviation is set and the inertia and rotation mapping of the instrument is calibrated. The samples are then spread out on the plate and the deviation from the cone plane geometry is set to 116 microns in viscosity measurement method (i) or to 105 microns in viscosity measurement method (ii). Advantageously, the excess sample is removed with a spatula to limit edge effects.

The higher the viscosity of the pharmaceutical formulation, the more localized the pharmaceutical formulation is in the tissue at the site of injection before forming a hydrogel.

In the pharmaceutical formulation of the disclosure, the mixture of Chitosan A and polysaccharide B are preferably present in the pharmaceutical formulation in dissolved form, meaning that at least 90%, preferably at least 95% of the mixture is in dissolved form, % expressed by weight of mixture in dissolved form, relative to the total weight of mixture in the pharmaceutical formulation.

The presence of the mixture of polysaccharides in dissolved form in the pharmaceutical formulation of the disclosure enables the pharmaceutical formulation to be under the form of a viscous solution sufficiently liquid to be injected via a wide range of needles commonly used in the medical field.

In other words, the pharmaceutical formulation of the disclosure is not under the form of a solid hydrogel, as it is in the case of implantable hydrogels.

### Biodegradable and Biocompatible Hydrogel

The polysaccharide mixture of the pharmaceutical formulation of the disclosure is capable of gelling in situ under physiological conditions, in particular at physiological pH and osmolarity, thus forming a hydrogel.

In the pharmaceutical formulation of the invention, the chitosan A, the polysaccharide B and the protein or combination of proteins are intimately mixed. Thus, the in situ gelling of the polysaccharide mixture results in the encapsulation of the protein or combination of proteins present in the pharmaceutical of the disclosure into the hydrogel.

The hydrogel formed in the tissue is biodegradable and biocompatible. Moreover, it may release the protein in a controlled, prolonged, and virtually constant manner, especially while avoiding a phenomenon of rapid initial release known as “burst release”.

According to the present disclosure, the term “gel” means a nonfluidic polymeric network swollen by a solvent.

According to the present disclosure, the expression “hydrogel” means a visco-elastic material comprising at least 60% by weight of water a physical or chemical gel. The hydrogel is a physical gel. In such physical gel, the driving forces of gel formation are not chemical reaction forming covalent bonds but more physical phenomena such as Van Der Waals interactions and/or hydrogen bonding and/or electrostatic interaction.

The hydrogel comprises approximately from 50% to 95% by weight of water, preferably from 50% to 80% by weight of water, relative to the total weight of hydrogel.

According to the present disclosure, the expression “biodegradable”, referring to the in situ formed hydrogel, means that the hydrogel naturally breaks down under physiological conditions, and in particular under the action of macrophages. The reactions involved during biodegradation may include hydrolysis reactions, that is to say the breaking of covalent bonds by reaction with water. These reactions may be catalyzed by the action of enzymes naturally present at the site of injection.

In an embodiment, more than 50%, preferably more than 80%, preferably more than 95% by weight of the hydrogel deteriorates in a period of time comprised between 10 and 100 days, preferably between 20 and 65 days, when the hydrogel is placed in physiological conditions, typically after subcutaneous injection in a subject.

In particular, the hydrogel of the disclosure may virtually release the protein over a period comprised between 10 and 100 days, preferably between 20 and 65 days from the day of administration of the pharmaceutical formulation of the invention.

A long degradation time advantageously enables to extend the time between two injections. This is particularly advantageous compared to prior art formulations for example with hyaluronidases, which must preferably be injected approximately every two weeks.

According to the present disclosure, the expression “biocompatible”, referring to the in situ formed hydrogel, means a hydrogel having the ability not to degrade the biological environment it is placed into. In particular, the hydrogel produces little or no inflammatory reactions even during a prolonged with biological environment.

In an embodiment, the hydrogel causes little or no inflammation over a period between 10 and 100 days, preferably between 20 and 65 days from the day of administration of the pharmaceutical formulation of the invention.

### Osmolarity

The pharmaceutical formulation of the disclosure preferably has a pH and/or an osmolarity close, but different from the physiological conditions, and in particular lower than the physiological conditions. When placed under physiological conditions, in contact with body fluids, the pharmaceutical formulation of the disclosure forms a gel by a change in pH and/or osmolarity by equilibration with physiological media.

The pharmaceutical formulation preferably has an osmolarity comprised between 50 and 600 mOsm/L, preferably comprised between 100 and 600 mOsm/L, more preferably between 250 and 450 mOsm/L, or between 50 and 300 mOsm/L, preferably between 50 and 250 mOsm/L.

According to the present disclosure, the osmolarity (also known as osmotic concentration) refers to the solute concentration per unit volume of solution. Osmolarity is similar to molarity but includes the total number of moles of dissolved species in solution. Osmolarity is defined as the number of osmoles (Osm) of solute per litre (L) of solution (osmol/L or Osm/L). An osmole is the amount of substance, which must be dissolved in order to produce an Avogadro's number of particles (6.0221×10²³). For substances, which do not dissociate, the molarity and the osmolarity will be the same, whereas for substances that are ionized the osmolarity will be the molarity multiplied by the number of dissociated parts, eg. for sodium chloride the osmolarity will be doubled. Osmolality is the number of osmoles of solute per kilogram of solvent. Physiological osmolarity is typically in the range of about 280 mOsm/L to about 310 mOsm/L, typically is about 300 mOsm/L.

The osmolarity of the pharmaceutical formulation of the disclosure is typically measured using an osmometer, such as a Löser Micro Osmometer MOD200 Plus from Camlab. Prior to measurement, zero is set using 50 μL of distilled water, and the instrument is then calibrated against 25 μL of a 300 mOsm/kg water standard. Samples are measured by taking an identical volume of 25 μL frozen to −6.0° C. Alternatively, zero is set using 15 μL of distilled water, and the instrument is then calibrated against 15 μL of a 300 mOsm/kg water standard. Samples are measured by taking an identical volume of 15 μL frozen to −6.2° C.

pH

The pharmaceutical formulation preferably has a pH comprised between 5.0 and 6.5, preferably between 5.0 and 6.4, more preferably between 5.5 and 6.0.

The pH of the pharmaceutical formulation of the disclosure is measured according to methods known to those skilled in the art, for example using a pH meter such as a Mettler Toledo SevenCompact S210 pH meter. According to the present disclosure, the expression “physiological pH” means a pH ranging between 6.8 and 7.6 typically of approximately 7.4.

### The Protein Contained in the Pharmaceutical Formulation

The pharmaceutical formulation of the disclosure comprises a therapeutically effective amount of a protein or of a combination of proteins.

According to the present disclosure, a “therapeutically effective amount” is the least concentration required to effect a measurable improvement or prevention of any symptom or a particular condition or disorder, to effect a measurable enhancement of life expectancy, or to generally improve patient quality of life. The therapeutically effective amount is dependent upon the specific biologically active molecule and the specific condition or disorder to be treated.

The content of protein or of combination of proteins in the pharmaceutical formulation is typically comprised between 1 g/l and 200 g/l, for example between 50 and 100 g/l of protein or combination of proteins.

Therapeutically effective amounts of many proteins, such as the antibodies described herein, are well known in the art. The therapeutically effective amounts of proteins not yet established or for treating specific disorders with known proteins, such as antibodies, to be clinically applied to treat additional disorders may be determined by standard techniques which are well within the craft of a skilled artisan, such as a physician.

According to the present disclosure, the term “protein” refers to a polymer of amino acid residues (natural or non-natural) linked together most often by peptide bonds. Proteins include naturally occurring proteins and recombinant proteins. The protein can be functionally linked (e.g., by chemical coupling, noncovalent association or otherwise) to one or more other molecules such as small molecules, polymer (e.g. polyethylene glycol) or other proteins. The term “protein” may be a single molecule or may be a multimolecular complex such as dimer, trimer or tetramer. They may also include single chain or multichain proteins such as antibodies. Disulfide linkages are commonly found in multichain proteins. The term protein may also apply to amino acid polymers in which one or more amino acids residues are an artificial chemical analogue or a corresponding naturally amino acid.

In the present disclosure, the protein molecular weight may be determined using standard methods known to one skilled in the art, including, but not limited to, mass spectrometry (e.g., ESI, MALDI, SDS page technique) or calculation from known amino acid sequences and glycosylation. Proteins can be naturally occurring or non-naturally occurring, synthetic, or semisynthetic. The protein or combinations of protein may in particular be derived from the human plasma.

The protein may have a molecular weight comprised between 10 kDa and 250 kDa, preferably between 20 kDa and 250 kDa, preferably between 100 kDa and 250 kDa, preferably between 120 kDa and 250 kDa, preferably between 150 kDa and 250 kDa.

In specific embodiments of the pharmaceutical formulation of the present disclosure, the protein preferably has a molecular weight comprised between 40 kDa and 250 kDa, advantageously between 100 kDa and 250 kDa, more advantageously between 120 kDa and 250 kDa, even more advantageously between 150 kDa to 250 kDa.

In specific preferred embodiments, the protein or combination of proteins is selected from the group consisting of an antibody, an enzyme, a fusion protein, and a combination thereof, in particular antibodies or their combination.

The pharmaceutical formulation of the disclosure is preferably devoid of recombinant human hyaluronidase PH20 (rHuPH20), preferably is devoid of enzyme able to degrade hyaluronan.

### Antibody

The protein or combination of proteins is preferably an antibody or a combination of antibodies.

According to the present disclosure, the term “antibody” refers to immunoglobulin molecules i.e., molecules that contain an antigen binding site that immunospecifically binds an antigen. As such, the term “antibody” encompasses not only whole antibody molecules, but also antibody fragments as well as variants (including derivatives) of antibodies and antibody fragments.

The combination of antibodies may be derived from the human plasma. In an embodiment, the antibodies derived from the plasma of unselected blood donors. Such antibodies are known are normal (i.e. nonspecific) immunoglobulins usually abbreviated HNI or HNIg. It may be used to provide antibodies to patient having primary immunodeficiency disorders (PIDs), hypogammaglobulinemia, primary immune thrombocytopenia, Guillain Barré syndrome, Kawasaki disease, multifocal motor neuropathy, chronic inflammatory demyelinating polyradiculoneuropathy, or secondary (i.e. acquired) immunodeficiency disorders. In another embodiment, the antibodies are derived from the plasma of selected blood donors. Such antibodies are known as “hyperimmune immunoglobulins”. They are prepared in a similar way as for normal human immunoglobulins, except that the blood donor has high titers of antibodies against a specific organism or antigen in their plasma. Some agents against which hyperimmune globulins may be used include hepatitis B, rabies, tetanus toxin, and varicella-zoster.

The antibody may be a monoclonal or a polyclonal antibody.

The term “monoclonal antibody”, according to the present disclosure, refers to an antibody obtained from a population of substantially homogeneous antibodies, i.e., the individual antibodies constituting the population bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The term “monoclonal” indicates the character of the antibody as being obtained from a substantially homogeneous population of antibodies and is not to be construed as requiring production of the antibody by any particular method. Specific examples of monoclonal antibodies herein include chimeric antibodies, humanized antibodies, and human antibodies.

According to the present disclosure, “chimeric” monoclonal antibodies are antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as long as they exhibit the desired biological activity. Chimeric antibodies of interest herein include “primatized” antibodies comprising variable domain antigen-binding sequences derived from a non-human primate (e.g. baboon, rhesus or cynomolgus monkey) and human constant region sequences. An example of a chimeric antibody is rituximab.

According to the present disclosure “Humanized” forms of non-human (e.g. murine) antibodies are chimeric antibodies that contain minimal sequence derived from non-human immunoglobulin. For the most part, humanized antibodies are human immunoglobulins in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody may optionally comprise at least a portion of an immunoglobulin constant region, typically that of a human immunoglobulin. An example of a humanized antibody is trastuzumab.

According to the present disclosure, a “full length antibody” is an antibody which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. Preferably, the full length antibody has one or more effector functions. Rituximab, and trastuzumab are examples of full-length antibodies.

According to the present disclosure, an “antibody fragment” comprises a portion of a full-length antibody, including the antigen binding and/or the variable region of the full-length antibody. Examples of antibody fragments include Fab, Fab′, F(ab′)2, and Fv fragments; single-domain antibodies. F(ab′)2, Fab, Fab′ and Fv are antigen-binding fragments that can be generated from the variable region of IgG and IgM. F(ab′)2 fragments contain two antigen-binding regions joined at the hinge through disulfides. F(ab′)2 fragments are generally void of most, but not all, of the Fc region. Fab′ fragments can be formed by the reduction of F(ab′)2 fragments. Fab′ fragments contain a free sulfhydryl group that may be alkylated or utilized in conjugation with an enzyme, toxin or other protein of interest. Fab′ fragments are derived from F(ab′)2; therefore, they may contain a small portion of Fc. Fab is a monovalent fragment that is produced from IgG and IgM, consisting of the VH, CH1 and VL, CL regions, linked by an intramolecular disulfide bond. Fv fragments refers to the smallest fragments produced from IgG and IgM that contains a complete antigen-binding site. Fv fragments have the same binding properties and similar three-dimensional binding characteristics as Fab. The VH and VL chains of the Fv fragments are held together by non-covalent interactions. A “Single-domain antibody”, also known as “nanobody”, is a monomeric antigen-binding fragment of an antibody which provides various advantages over other antibody fragments as “building blocks” for bsAbs. They occur in nature as the antigen-binding portion of heavy chain antibodies in camelid species (called VHH) and cartilaginous fish (called VNAR) or can be generated from conventional IgGs by obtaining or engineering monomeric, stable VH or VL domains.

Exemplary antibodies which can be formulated according to the present disclosure include, but are not limited to Abatacept, Adalimumab, Alemtuzumab, Alirocumab, Amivantamab, Anifrolumab, Atezolizumab, Avelumab, Balstilimab, Basiliximab, Belatacept, Belimumab, Benralizumab, Besilesomab, Bevacizumab, Bezlotoxumab, Bimekizumab, Blinatumomab, Brodalumab, Burosumab, Canakinumab, Carotuxomab, Cemiplimab, Cetuximab, Concizumab, Crizanlizumab, Daratumumab, Denosumab, Dinutuximab Beta, Dostarlimab, Dupilumab, Durvalumab, Eculizumab, Elotuzumab, Epratuzumab, Erenumab, Evolocumab, Fab Ig Antidigitalique Ovin, Fremanezumab, Galcanezumab, Golimumab, Guselkumab, Ibalizumab, Ig Anti Human Lymphocyte (Lapine), Ig Anti Humane Thymocyte (Equine), Ig Anti Human Thymocyte (Lapine), Infliximab, Iph 4102, Ipilimumab, Isatuximab, Ixekizumab, Lacutamab, Lag525, Lanadelumab, Mcla-128, Mepolizumab, Natalizumab, Naxitamab, Nimotuzumab, Nivolumab, Obinutuzumab, Ocrelizumab, Ofatumumab, Omalizumab, Palivizumab, Panitumumab, Pembrolizumab, Pertuzumab, Ramucirumab, Ranibizumab, Reslizumab, Risankizumab, Rituximab, Sarilumab, Secukinumab, Seribantumab, Siltuximab, Spartalizumab, Tafasitamab, Teprotumumab, Tildrakizumab, Tocilizumab, Tralokinumab, Trastuzumab, Urelumab, Ustekinumab, Vedolizumab, Zalifrelimab, and combinations thereof.

Exemplary combination of antibodies which can be formulated according to the present disclosure include, but are not limited to Casirivimab and Imdevimab, a combination of Pertuzumab and Trastuzumab or a combination of Tixagevimab and Cilgavimab.

The antibody can be functionally linked (e.g., by chemical coupling, genetic fusion, noncovalent association or otherwise) to one or more other molecules to increase half-life or stability or otherwise improve the antibody.

For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. The antibody may be functionally linked to poly(ethylene glycol), for example the antibody is Certolizumab Pegol. The antibody can be functionally linked to chelating agents such as DOTA (1,4,7,10-tetraazacyclododecane-N,N′,N″,N′″-tetracetic acid), NOTA (1,4,7-triazacyclononane-1,4,7-triacetic acid), NODAGA (1,4,7-triazacyclononane-1-glutaric acid-4,7-diacetic acid, DOTAGA (2-(4,7,10-tris(carboxymethyl)-1,4,7,10-tetraazacyclododecan-1-yl)pentanedioic acid), DOTAM (1,4,7,10-tetrakis(carbamoylmethyl)-1,4,7,10 tetraazacyclododecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N′″-tetraacetic acid), TETAM (1,4,8,11-tetraazacyclotetradecane-N,N′,N″,N′″-tetrakis(carbamoyl methyl), DTPA (diethylene triaminopentaacetic acid), DFO (deferoxamine), Bz-DFO, bispitine ([3,7]-diazabicyclo[3.3.1]nonane) preferably is functionally linked to a chelating agent selected from DOTAGA, DFO, Bz-DFO, DOTAM, DTPA and bispitine,

The antibody may also be functionally linked to a cytotoxic agent such as calicheamicin, monomethyl auristatin F (MMAF also known as mafodotin), monoethyl auristatin E (MMAE also known as vedotin), Emtamsine (DM1), Exatecan derivative (Dxd), N-acetyl-gamma calicheamicin thereby forming antibody-drug conjugates. Exemplary antibody-drug conjugate (ADCs) which can be formulated according to the present disclosure include, but are not limited to, Belantamab Mafodotine, Brentuximab Vedotin, Depatuxizumab Mafodotine, Enfortumab Vedotin, Gemtuzumab Ozogamicine, Inotuzumab Ozogamicin, Moxetumomab Pasudotox, Sacituzumab Govitecan, Trastuzumab Deruxtecan, Trastuzumab Emtansine.

The antibody may be functionally linked to one or more antibodies or antibody fragments, to generate a bispecific or a multi-specific molecule. Exemplary bispecific antibodies which can be formulated according to the present disclosure include, but are not limited to Glofitamab, KN046, 1B1318, 1B1318, Emicizumab, Epcoritamab, Tebotelimab, Tebentafusp, Teclistamab, Faricimab, Amivantamab, Mosunetuzumab, Zanidatamab, Flotetuzumab, APV0436, Zenocutuzumab, TNB383B, and combinations thereof. In an embodiment, the bispecific antibody that is formulated is Epcoritamab.

In an embodiment, the protein or combination of proteins is an antibody or combination of antibodies which binds an antigen selected from the group of HER2 and CD20. CD20 antibodies may be used for therapy of B cell malignancies (such as non-Hodgkin's lymphoma or chronic lymphocytic leukemia) or autoimmune diseases (such as rheumatoid arthritis and vasculitis); HER2 antibodies may be used for cancer therapy (such as breast cancer or gastric cancer). The phrases “an antibody which binds an antigen”, “an antibody recognizing an antigen” and “an antibody having specificity for an antigen” have the same meaning and will be used equally herein.

In one embodiment the antibody which is formulated binds HER2. In one preferred embodiment, the antibody which is formulated is trastuzumab.

In one embodiment the antibody which is formulated binds CD20. In one preferred embodiment, the antibody which is formulated is rituximab.

In one embodiment the antibody which is formulated binds HER2. In one preferred embodiment, the antibody which is formulated is pertuzumab.

In one embodiment the antibody which is formulated binds CD38. In one preferred embodiment, the antibody which is formulated is daratumumab.

In one embodiment the combination of antibodies which is formulated is a combination of antibodies which bind two distinct epitopes of HER2. In one preferred embodiment, it includes a combination of trastuzumab and pertuzumab.

### Excipients

The pharmaceutical formulation of the disclosure may further comprise one or more pharmaceutically acceptable excipients selected from solvents, stabilizers, surfactants, buffering agents, antimicrobial preservatives, protectants, antioxidants, chelating agents, and bulking agents.

According to the present disclosure, a “solvent” is any pharmaceutically acceptable (i.e., safe and non-toxic for administration to a human or another mammal) and useful ingredient for the preparation of a liquid formulation, such as an aqueous formulation. Exemplary solvents include water such as sterile water for injection (WFI) or bacteriostatic water for injection (BWFI), pH buffered solution (e.g., phosphate-buffered saline), sterile saline solution, Ringer's solution or dextrose solution, and combinations thereof. Preferably, the solvent is sterile water for disclosure or bacteriostatic water for injection (BWFI).

According to the present disclosure, stabilizers are compounds increasing protein stability, especially against unfolding and aggregation. Preferably, the stabilizer is admitted by the authorities as a suitable additive or excipient in pharmaceutical formulations.

The stabilizer may be a saccharide. A “saccharide” herein comprises the general composition (CH₂O)ₙ and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc.

Preferably, the formulation comprise a non-reducing disaccharide as a stabilizing agent, such as a saccharide selected from the group of trehalose (e.g. in the form of α,α-trehalose dihydrate) and sucrose.

The concentration of the stabilizer in the pharmaceutical formulation of the disclosure is preferably comprised between 1 and 500 mM, 15 and 250 mM, or 150 and 250 mM, or is about 210 mM.

According to the present disclosure, a “surfactant” refers to a surface-active agent. Surfactants are generally added in protein formulations in order to reduce the exposure of hydrophobic regions and so decreasing protein-protein interactions and interface-induced aggregation, also prevented by competition for adsorption sites.

Examples of surfactants herein include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; polyethylglycol, polypropyl glycol, and copolymers of ethylene and propylene glycol (e.g. Pluronics, PF68 etc). Other examples of pharmaceutically acceptable surfactants include polyoxyethylen-sorbitan fatty acid esters (Tween), polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), and sodium dodecyl sulphate (SDS). Most suitable polyoxyethylenesorbitan-fatty acid esters are polysorbate 20, (sold under the trademark Tween 20™) and polysorbate 80 (sold under the trademark Tween 80™).

Most suitable polyethylene-polypropylene copolymers are those sold under the names Pluronic® F68 or Poloxamer 188™. Most suitable polyoxyethylene alkyl ethers are those sold under the trademark Brij™. Most suitable alkylphenol-polyoxyethylene ethers are sold under the trade name Triton-X.

The surfactant is preferably a nonionic surfactant, preferably a polysorbate, e.g. selected from the group of polysorbate 20, polysorbate 80 and polyethylene polypropylene copolymer.

The concentration of surfactant in the pharmaceutical formulation of the disclosure is preferably comprised between 0.01 and 0.1% (w/v), or 0.01 an 0.08% (w/v), or 0.025 and 0.075% (w/v).

According to the present disclosure, the term “buffering agent” refers to an agent which provides that the solution comprising it resists changes in pH by the action of its acid/base conjugate components. Examples of buffering agents that will control the pH in this range include acetate, succinate, gluconate, histidine, citrate, glycylglycine and other organic acid buffers.

A suitable buffer in the present disclosure is a histidine buffer.

A “histidine buffer” is a buffer comprising the amino acid histidine. Examples of histidine buffers include histidine chloride (e.g. L-histidine hydrochloride monohydrate), histidine acetate, histidine phosphate, histidine sulfate.

A “preservative” is a compound which can be added to the formulations herein to reduce contamination by and/or action of bacteria, fungi, or another infectious agent. The addition of a preservative may, for example, facilitate the production of a multi-use (multiple-dose) formulation. Examples of potential preservatives include octadecyldimethylbenzylammonium chloride, hexamethonium chloride, benzalkonium chloride (a mixture of alkylbenzyldimemylammonium chlorides in which the alkyl groups are long-chained), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and w-cresol.

A “protectant”, as generally used herein, is a substance which, when combined with a protein, significantly reduces chemical and/or physical instability of the protein upon lyophilization and/or subsequent refrigerated storage.

Exemplary protectants include sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or poly(propylene glycol); and combinations thereof. Additional exemplary of protectants include gelatin, dextrins, modified starch, and carboxymethyl cellulose. Preferred sugar alcohols are those compounds obtained by reduction of mono- and di-saccharides, such as lactose, trehalose, maltose, lactulose, and maltulose. Additional examples of sugar alcohols are glucitol, maltitol, lactitol and isomaltulose.

The protectant may be added to the pre-lyophilized formulation in a “lyoprotecting amount.” This means that, following lyophilization of the protein in the presence of lyoprotecting amount of the protectant, the protein essentially retains its physical and chemical stability and integrity.

An “antioxidant”, as generally used herein is a pharmaceutically acceptable excipient generally used to limit oxidation reactions and maintain the stability and safety of proteins. Examples of antioxidants are ascorbic acid, sodium metabisulfite, histamine, methionine, ascorbic acid, glutathione, vitamin E, polyethylenimine.

The antioxidant is preferably methionine, in particular L-methionine.

The antioxidant concentration in the pharmaceutical formulation of the disclosure is preferably comprised between 5 and 25 mM, more preferably between 5 and 15 mM.

A “chelating agent”, as generally used herein in the context of the excipients, is a pharmaceutically acceptable excipient generally used to maintain the stability of proteins.

Examples of chelating agent include edetate disodium, diethylenetriamine penta-acetic acid, citric acid, hexaphosphate, thioglycolic acid, zinc.

A “bulking agent,” as generally used herein, is a pharmaceutically acceptable excipient generally used to add mass to a lyophilized mixture and contributes to the physical structure of the lyophilized cake (e.g. facilitates the production of an essentially uniform lyophilized cake which maintains an open pore structure). Exemplary bulking agents include mannitol, glycine, lactose, modified starch, polyethylene glycol), and sorbitol.

### Specific Pharmaceutical Formulations

In an embodiment, the pharmaceutical formulation of the disclosure preferably comprises:
A protein or a combination of proteins, preferably an antibody or a combination of antibodies as defined herein, in particular trastuzumab, rituximab, pertuzumab, daratumumab, or a combination of trastuzumab and pertuzumab,
a mixture of polysaccharides as defined herein,
and preferably at least one of
   a buffering agent, a stabilizer, or an antioxidant as defined herein.

In an embodiment, the pharmaceutical formulation of the disclosure preferably comprises:
between 1 g/l and 200 g/l, preferably between 100 et 150 g/l of an antibody or a combination of antibodies, in particular trastuzumab, rituximab, pertuzumab, daratumumab, or a combination of trastuzumab and pertuzumab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B,
and preferably at least one of:
   1 to 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   1 to 500 mM of a stabilizer, preferably of trehalose such as α,α-trehalose dihydrate, or
   5 to 25 mM of an antioxidant, preferably of methionine.

In an embodiment, the pharmaceutical formulation of the disclosure preferably comprises:
between 1 g/l and 200 g/l, preferably between 100 et 150 g/l of trastuzumab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B,
and preferably at least one of:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

In an embodiment, the pharmaceutical formulation of the disclosure preferably comprises:
between 1 g/l and 200 g/l, for example between 5 et 20 g/l, preferably between 100 et 150 g/l of rituximab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B
and preferably at least one of:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

In an embodiment, the pharmaceutical formulation of the disclosure preferably comprises:
between 1 g/l and 200 g/l for example between 30 et 50 g/l or about 40 g/l, preferably between 100 et 150 g/l of trastuzumab,
between 1 g/l and 200 g/l, for example between 50 et 100 g/l or about 80 g/l, preferably between 100 et 150 g/l of pertuzumab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B,
and preferably at least one of:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

In an embodiment, the pharmaceutical formulation of the disclosure preferably comprises:
between 1 g/l and 200 g/l for example between 50 et 100 g/l, preferably between 100 et 150 g/l of pertuzumab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B,
and preferably at least one of:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

In an embodiment, the pharmaceutical formulation of the disclosure preferably comprises:
between 1 g/l and 200 g/l for example between 50 et 100 g/l, preferably between 100 et 150 g/l of daratumumab,
between 5 and 200 g/l, preferably between 10 and 200 g/l, more preferably between 10 and 150 g/l of chitosan A,
between 10 and 200 g/l, preferably between 20 and 100 g/l of statistic polysaccharide B,
and preferably at least one of:
   between 1 and 100 mM of a buffering agent, preferably a histidine buffer, more preferably a histidine chloride buffer such as L-histidine hydrochloride monohydrate, to provide a pH between 5.0 and 6.5, preferably between 5.0 and 6.4, more preferably between 5.5 and 6.00,
   between 1 and 500 mM of a stabilizer, preferably of trehalose such as α,α-trehalose dihydrate, or
   between 5 and 25 mM of an antioxidant, preferably of methionine.

### Stability in Time

The pharmaceutical formulation of the disclosure may be conserved for a long period of time, typically over a period of time of at least 6 months preferably over a period of time comprised between 6 months and one year.

### Ready-to-Use

In an embodiment, the pharmaceutical formulation of the disclosure is in the form of a ready-to-use (i.e. ready-to-administer) injectable formulation containing the polysaccharide mixture, the protein or combination of proteins and one or more pharmaceutically acceptable excipients as defined in the present disclosure.

In that embodiment, the pharmaceutical formulation may, for example, be supplied in a pre-filled syringe.

### Preparation Method

In a second aspect, the disclosure relates to a process for preparing a pharmaceutical formulation as defined in the first aspect of the disclosure comprising at least a step of mixing a polysaccharide mixture, a protein, or a combination of proteins and one or more pharmaceutical excipients as defined in the first aspect of the disclosure.

In an embodiment, process comprises the following steps:
a) providing a solution containing a polysaccharide mixture as defined in the first aspect of the disclosure,
b) providing a solution containing a protein or a combination of protein as defined in the first aspect of the disclosure,
c) mixing the solution of step a) and solution of step b) to obtain an homogenous solution,
d) optionally, adjusting the osmolarity of the solution obtained in step c) to provide an osmolarity comprised between 50 and 300 mOsm/L, preferably between 50 and 250 mOsm/L,
e) optionally, adjusting the pH of the solution obtained in step c) between 5.0 and 6.5, preferably between 5.0 and 6.4, more preferably between 5.5 and 6.0.

The solutions of step a) and step b) are preferably aqueous solution.

The solution of step b) may be prepared by dissolving a protein in powder form e.g. lyophilized form, typically by mixing the powder and a solvent in particular a solvent suitable for injection to form a solution containing a protein or a combination of proteins.

Step c) may be carried out by centrifugation. For example, the centrifugation may be carried out between 2000 and 8000 rpm during between 5 and 30 minutes, preferably about 5000 rpm during 10 minutes or between 50 and 200 rpm during 1 and 4 hours, preferably about 100 rpm during 1 hour. Alternatively, centrifugation may be carried out after step c) to remove air bubbles from the solution obtained in step c).

In an embodiment, step c) may be carried out by a static mixer. In that embodiment, the solution of step a) is contained in a first container and the solution of step b) is contained in a second container, said containers being connected to a static mixer, to provide a system for mixing the solution of step a) and the solution of step b) by injection of the contents of one solution into the other and vice versa. Such injection may be repeated between 100 and 500 times, typically between 100 and 300 times.

Step c) is preferably carried out in a static mixer.

In an embodiment, step c) may be carried out by a static mixer, by mixing directly the solution of step a) and the solution of step b) in a reactor.

The optional osmolarity adjustment of step d) may be carried out by appropriate dialysis of the solution obtained in step c), for example against water or a solution containing a salt such as NaCl, optionally maintaining the pH by adding a base solution.

The optional pH adjustment of step e) may be carried out by appropriate addition of a base or an acid, preferably of a base such as NaOH.

Steps d) and e) may be carried out in any order, i.e. step d) precedes step e), or step e) precedes step d), or may be performed concomitantly. For example, the osmolarity may be adjusted by dialysis while maintaining the pH or vice versa.

### Kit of Parts

A third aspect of the disclosure relates to a kit, comprising:
at least one first container containing the chitosan A and the statistic polysaccharide B i.e. the polysaccharide mixture as defined in the first aspect of the disclosure,
at least one second container containing the protein or combination of proteins as defined in the first aspect of the disclosure.

The kit is for preparing a pharmaceutical formulation according to the present disclosure, in particular a ready-to-use injectable formulation according to the present disclosure.

In an embodiment, the polysaccharide mixture of the first container is in the form of a powder or of a liquid solution.

In an embodiment, the protein or combination of proteins of the second container is in the form of a powder or of a liquid solution.

In an embodiment, the polysaccharide mixture of the first container and the protein or combination of proteins of the second container are in the form of a powder or of a liquid solution.

In an embodiment, the first and second containers may be connected to a static mixer which provides a mechanism for mixing the polysaccharide mixture and the protein or combination of proteins. For example, the first and second connector may comprise a Luer lock adapter and be connected via a Luer lock connector.

The first and/or second container are preferably syringes, more preferably ready-to use syringes. In an embodiment the first and second containers are Luer lock vials or syringes connected by a Luer lock connector.

The kit of the disclosure allows to prepare the pharmaceutical composition from any source of sterile commercial protein, resulting in a great versatility.

Besides, the kit of the disclosure allows the formulation to be prepared shortly before injection.

If the kit comprises excipients other than solvent and buffering agents are as stabilizer, these are preferably in the second container.

### Administration

The pharmaceutical formulation of the disclosure is preferably suitable for injection, preferably subcutaneous injection.

The “injectability” or “syringeability,” as generally used herein, refers to the injection performance of a pharmaceutical formulation through a syringe equipped with an 18-32, preferably 20-27 gauge needle.

According to the present disclosure, “subcutaneous injection” means an injection administered into the subcutis, the layer of skin directly below the dermis and epidermis, collectively referred to as the cutis. Subcutaneous administration may be abbreviated as SC, SQ, sub-cu, sub-Q, SubQ, or subcut.

The subcutaneous injection may be performed at any area suitable for subcutaneous injection, such as the thighs, the abdomen particularly in its umbilical region, the upper arm, in particular the posterior or lateral aspect of the lower part of the upper arm, the back, the lower loins, and the buttocks, in particular the upper outer area of the buttocks.

Several subcutaneous injections can be done at the same time in different areas suitable for subcutaneous injection.

The subcutaneous injection volume may be comprised between 1 and 20 ml, preferably between 2 and 15 ml.

As shown in the experimental part of the application, the pharmaceutical composition injection volume does not interfere with the degradation kinetic of the hydrogel formed after injection. Besides, it is also shown that the scaffold of the hydrogel formed after injection and hence the release of the protein entrapped can be modulated depending e.g. on the chitosan A/statistic polysaccharide B weight ratio.

In one embodiment, the subcutaneous injection volume is comprised between 1 and 5 ml, preferably between 1 and 3 ml, preferably is about 2 ml. In that embodiment, the subcutaneous injection is preferably performed in the subcutaneous tissue of the thighs, the upper arm, the back and the lower loins.

In one embodiment, the subcutaneous injection volume is comprised between 5 and 20 ml, preferably between 5 or 10 ml or between 10 and 20 ml, more preferably is about 15 ml. In that embodiment, the subcutaneous injection is preferably performed in the subcutaneous tissue of the abdomen.

The pharmaceutical formulation may also be administrated by intraperitoneal injection, intra-articular injection, intrathecal injection, or intra ocular injection.

According to the present disclosure, “intraocular injection” (also known as intravitreal injections) means a route of administration via an injection inside of the eye via the vitreous i.e. the gel-like substance that fills the eye.

According to the present disclosure, “intra-articular injection” means a route of administration via an injection into a joint. intra-articular injection may be useful in chemotherapy, as well as in treating arthritis.

According to the present disclosure, “intrathecal administration” means a route of administration via an injection into the spinal canal or into the subarachnoid space so that it reaches the cerebrospinal fluid (CSF). Intrathecal administration may be useful chemotherapy.

The pharmaceutical formulation may also be administered by intratumoral injection.

According to the present disclosure, “intratumoral injection” means a direct injection into a tumor. Intratumoral injections can be considered for any tumor where the primary lesion or its metastases are accessible either percuteanously (i.e. needle-puncture of the skin) via direct injection or via specific procedures such as colonoscopy, cystoscopy, bronchoscopy, thoracoscopy, coelioscopy, or even surgery.

The injection may be administered with a 18-32-gauge needle, preferably a 20-27 gauge needle. The formulations can be administered using a small gauge needle, for example, between 20 and 30 gauge, typically 27, 28 29, or 30 gauge.

In an embodiment, the pharmaceutical formulation causes no significant inflammation when administered not more than twice daily, once daily, twice weekly, once weekly, once every two weeks or once monthly. The pharmaceutical formulation of the disclosure is preferably administered once every two weeks causing no significant inflammation at the site of injection.

In an embodiment, the pharmaceutical formulation exhibits increased bioavailability compared to formulation comprising the same protein when administered via subcutaneous injection. “Bioavailability” refers to the extent and rate at which the protein such as the antibody, reaches circulation or the site of action. One way of measuring the bioavailability is by comparing the “area under the curve” (AUC) in a plot of the plasma concentration as a function of time.

The AUC can be calculated, for example, using the linear trapezoidal rule. “AUCₒ”, according to the present disclosure, refers to the area under the plasma concentration curve from time zero to a time where the plasma concentration returns to baseline levels. “AUCₒ₋ₜ”, according to the present disclosure, refers to the area under the plasma concentration curve from time zero to a time, t, later, for example to the time of reaching baseline. The time is typically be measured in days, although hours can also be used as will be apparent by context. For example, the AUC can be increased by more than 10%, 20%, 30%, 40%, or 50% as compared to the otherwise same formulation without the viscosity-lowering water soluble organic dye(s) and administered in the same way.

According to the present disclosure, “tₘₐₓ” refers to the time after administration at which the plasma concentration reaches a maximum.

According to the present disclosure, “Cₘₐₓ” refers to the maximum plasma concentration after dose administration, and before administration of a subsequent dose.

According to the present disclosure, “Cₘᵢₙ” refers to the minimum plasma concentration after dose administration, and before administration of a subsequent dose.

In an embodiment, an advantageous characteristic of the pharmaceutical formulation of the disclosure is that the tₘₐₓ after injection, preferably after SC injection, increases relative to the tₘₐₓ of a formulation wherein the polysaccharide mixture is absent, typically a pharmaceutical containing a recombinant human hyaluronidase PH20 (rHuPH20), or more generally an enzyme able to degrade hyaluronan, this increase being greater than or equal to 1.5×, preferably greater than equal or 3×.

Without wishing being bound by any theory, the inventors believe that the Tₘₐₓ increase observed in Example 8 confirms a slower release of the trastuzumab when compared to the formulation ROCHE HERCEPTIN® SC.

The pharmaceutical formulation of the disclosure can allow for greater flexibility in dosing and decreased dosing frequencies compared to pharmaceutical formulations without the polysaccharide mixture. For example, by increasing the dosage administered per injection multiple-fold, the dosing frequency can in some embodiments be decreased from once every 2 weeks to once every 6 weeks.

### Therapeutic Use

In a fourth aspect, the disclosure relates to the pharmaceutical formulation of the first aspect of the disclosure or the kit of the second aspect of the disclosure for use as a medicament, preferably for use in the treatment of cancer.

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma and islet cell cancer), mesothelioma, schwannoma (including acoustic neuroma), meningioma, adenocarcinoma, melanoma, and leukemia or lymphoid malignancies (including waldenstrom macroglobulinemia), multiple myeloma or myeloid neoplasms. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

The pharmaceutical formulation of the first aspect of the disclosure or the kit of the second aspect of the disclosure may also be for use in the treatment of nonmalignant diseases in particular nonmalignant diseases selected from autoimmune disease (e.g. psoriasis); endometriosis; scleroderma; restenosis; polyps such as colon polyps, nasal polyps or gastrointestinal polyps; fibroadenoma; respiratory disease; cholecystitis; neurofibromatosis; polycystic kidney disease; inflammatory diseases; skin disorders including psoriasis and dermatitis; vascular disease; conditions involving abnormal proliferation of vascular epithelial cells; gastrointestinal ulcers; Menetrier's disease, secreting adenomas or protein loss syndrome; renal disorders; angiogenic disorders; ocular disease such as age related macular degeneration, presumed ocular histoplasmosis syndrome, retinal neovascularization from proliferative diabetic retinopathy, retinal vascularization, diabetic retinopathy, or age related macular degeneration; bone associated pathologies such as osteoarthritis, rickets and osteoporosis; damage following a cerebral ischemic event; fibrotic or edemia diseases such as hepatic cirrhosis, lung fibrosis, carcoidosis, throiditis, hyperviscosity syndrome systemic, Osier Weber-Rendu disease, chronic occlusive pulmonary disease, or edema following burns, trauma, radiation, stroke, hypoxia or ischemia; hypersensitivity reaction of the skin; diabetic retinopathy and diabetic nephropathy; Guillain-Barre syndrome; graft versus host disease or transplant rejection; Paget's disease; bone or joint inflammation; photoaging (e.g. caused by UV radiation of human skin); benign prostatic hypertrophy; certain microbial infections including microbial pathogens selected from adenovirus, hantaviruses, Borrelia burgdorferi, Yersinia spp. and Bordetella pertussis; thrombus caused by platelet aggregation; reproductive conditions such as endometriosis, ovarian hyperstimulation syndrome, preeclampsia, dysfunctional uterine bleeding, or menometrorrhagia; synovitis; atheroma; acute and chronic nephropathies (including proliferative glomerulonephritis and diabetes-induced renal disease); eczema; hypertrophic scar formation; endotoxic shock and fungal infection; familial adenomatosis polyposis; neurodedenerative diseases (e.g. Alzheimer's disease, AIDS-related dementia, Parkinson's disease, amyotrophic lateral sclerosis, multiple sclerosis, retinitis pigmentosa, spinal muscular atrophy and cerebellar degeneration); myelodysplastic syndromes; aplastic anemia; ischemic injury; fibrosis of the lung, kidney or liver; T-cell mediated hypersensitivity disease; infantile hypertrophic pyloric stenosis; urinary obstructive syndrome; psoriatic arthritis; and Hasimoto's thyroiditis. Exemplary nonmalignant indications for therapy herein include psoriasis, endometriosis, scleroderma, vascular disease (e.g. restenosis, artherosclerosis, coronary artery disease, or hypertension), colon polyps, fibroadenoma or respiratory disease (e.g. asthma, chronic bronchitis, bronchieactasis or cystic fibrosis), inflammatory bowel diseases (IBD), primary immunodeficiency disorders (PIDs), hypogammaglobulinemia, primary immune thrombocytopenia, Guillain Barré syndrome, Kawasaki disease, multifocal motor neuropathy, chronic inflammatory demyelinating polyradiculoneuropathy.

In the present disclosure, the term“treatment” encompasses curative treatment or preventive treatment. The term “curative treatment” refers to a treatment that aims to cure a disease or to improve symptoms associated with a disease. The term “preventive treatment” refers to a treatment that aims to prevent a disease or to improve symptoms associated with a disease.

In an embodiment, the pharmaceutical formulation of the first aspect of the disclosure or the kit of the second aspect of the disclosure comprises an antibody or combination of antibodies.

In an embodiment, the pharmaceutical formulation of the first aspect of the disclosure or the kit of the second aspect of the disclosure comprises an antibody or combination of antibodies which binds an antigen selected from the group of HER2 such as trastuzumab, pertuzumab or a combination thereof and is for use in treating of breast cancer, gastric cancer, B cell malignancies (such as non-Hodgkin's lymphoma or chronic lymphocytic leukemia) or autoimmune diseases (such as rheumatoid arthritis and vasculitis).

In an embodiment, the pharmaceutical formulation of the first aspect of the disclosure or the kit of the second aspect of the disclosure comprises an antibody or combination of antibodies which binds an antigen selected from the group of CD20 antibody such as rituximab and is for use in treating rheumatoid arthritis, non-Hodgkin's lymphoma (NHL), leukemia such as chronic lymphocytic leukemia, granulomatosis with polyangiitis (Gpa or Wegener's disease), microscopic polyangiitis (Mpa) or Pemphigus vulgaris.

In an embodiment, the pharmaceutical formulation of the first aspect of the disclosure or the kit of the second aspect of the disclosure comprises an antibody or combination of antibodies which binds an antigen selected from the group of CD38 antibody such as daratumumab and is for use in treating multiple myeloma or myeloid neoplasms.

In a fifth aspect, the disclosure relates to a method of treating a cancer or a nonmalignant disease as defined in the fourth aspect of the disclosure in a subject in need thereof, said method comprising subcutaneously administering a therapeutically effective amount of pharmaceutical formulation of the disclosure in said subject.

In a sixth aspect, the disclosure relates to the use of the pharmaceutical formulation as disclosed herein in the manufacture of a medicament for the treatment of a cancer or a nonmalignant disease as defined in the fourth aspect of the disclosure.

According to the present disclosure, the singular forms “a”, “an”, and “the” include both singular and plural referents unless the context clearly dictates otherwise.

According to the present disclosure, the term “about” or “approximately” herein when referring to a measurable value such as a parameter, an amount, a temporal duration, and the like, is meant to encompass variations of +/−5% or less, more preferably +/−2%, and more preferably +/−1% or less of and from the specified value, insofar such variations are appropriate to perform in the disclosed invention.

### EXAMPLES

The present disclosure is further illustrated by the following examples.

The materials used in the Examples are listed below:

### Non-Functionalized Precursor Chitosan (Chitosan A)

The synthesis of MEX-CD2-I-tmb involves a non-functionalized precursor chitosan (Chitosan A) of medical grade and animal origin. The weight and number average molar masses (Mw=2,583.105 g/mol, Mn=1,323.105 g/mol respectively) were determined by size exclusion chromatography coupled with refractive index and multi-angle laser light scattering measurements (Schatz et al. Biomacromolecules, 2003; 4(3):641-8). The degree of acetylation (proportion of N-acetyl-D-glucosamine unit) of such crude chitosan (Chitosan A) was determined by 1H NMR spectroscopy using the Hirai method (Hirai et al., Polymer Bulletin, 26, 87-94.1991) and is estimated to be 6±0.5%.

### Polysaccharide MEX-CD2 (Polysaccharide B)

This synthesis of MEX-CD2-I-tmb also involves a polysaccharide MEX-CD2 (polysaccharide B) obtained after chemical modification of chitosan A with DOTA-GA anhydride, the synthesis of which is described in a previous patent application FR2110474. Briefly, 60 g of chitosan were introduced into a 10 L reactor with 4 L of ultrapure water and 50 mL of acetic acid, then the mixture was placed under mechanical stirring at 500 rpm. After complete dissolution of the chitosan (3 h), 4 L of 1,2-propanediol was added to the medium and the mixture was kept under stirring until homogenization (2 h). 120 g of DOTA-GA anhydride was then added and the mixture was kept under stirring overnight. The synthesis product was then purified by tangential filtration using the Sartoflow® Advanced device with a Sartocon® Slice PESU cassette (polyethersulfone membranes; cut-off: 100 kDa; filtration area: 0.1 m²) according to a diafiltration-concentration model against 200 L of a 0.1M acetic acid solution, then 200 L of a 5 mM acetic acid solution. The degree of acetylation of polysaccharide B is identical to that of the starting chitosan A and the degree of substitution of polysaccharide B (proportion of N-DOTAGA-D-glucosamine unit) was determined by the copper chelation method (Natuzzi et al., Nature Scientific Reports, 2021, 11, 19948) and is estimated to be 15±0.5%.

### Trastuzumab

The synthesis of MEX-CD2-I-tmb involves the antibody trastuzumab. Trastuzumab was obtained from the commercial Trazimera® powder for concentrate for solution for infusion. One vial contains 420 mg of trastuzumab, and excipients, namely L-histidine hydrochloride monohydrate, L-histidine, sucrose, and polysorbate 20 (E 432).

The in vivo experiments involve a HERCEPTIN® SC formulation comprising trastuzumab and recombinant human hyaluronidase (rHuPH20) as a key excipient. HERCEPTIN® SC formulation is under the form of a 600 mg/5 mL single-dose vial. It is a ready to use solution for injection which does not need to be diluted.

### Example 1: Preparation of a Formulation MEX-CD2-I-tmb (Also Referred to as MEX-CD2-I-HER2) in Accordance with the Invention, According to a Method 1

According to a method (1), the preparation of MEX-CD2-I-tmb was carried out from a viscous solution of composition referred to as MEX-CD2-I solution with a ratio α/β % where α is the mass concentration (w/v) of chitosan A and β the mass concentration (w/v) of polysaccharide B in the considered formulation.

Briefly, 3.0 g of polysaccharide B (MEX-CD2) and 1.5 g of chitosan A were dispersed in 28.93 mL of milli-Q water and 1.07 mL of ultrapure acetic acid are added under mechanical stirring at 50 rpm in a 50 mL reactor. The mixture was left under stirring for 24 hours until complete dissolution and homogenization of the medium.

The solution obtained was recovered and introduced into a suitable fluid dispenser, then centrifuged at 4000 rpm for 10 minutes to obtain an air bubble-free solution comprising the composition. A MEX-CD2-I solution was obtained.

In this method (1), 1.5 mL of this 5/10% MEX-CD2-I solution was thoroughly mixed in a reactor with 1.5 mL of a 420 mg Trazimera® solution (a commercial trastuzumab solution formulated with excipients containing a histidine buffer and a surfactant) previously reconstituted to 250 g/L in 1.68 mL of milli-Q water.

The mixture was centrifuged at 5000 rpm for 10 minutes and then re-mixed to homogenize the solution. This step was repeated three times until a homogeneous opaque white viscous solution was obtained.

The mixture was then introduced into a SpectraPor® Float-A-Lyzer® dialysis tube (cut-off: 8-10 kDa) and dialyzed for 24 hours against milli-Q water, maintaining the pH at 6.5 by adding a 0.1 mol/L NaOH aqueous solution. The mass contained in the dialysis tube was checked by weighing before dialysis. The dialysis solution was then changed to an 8 g/L sodium chloride solution where the pH was still maintained at 6.5 by adding a 0.1 mol/L NaOH solution and dialysis was continued for 48 hours. At the end of the dialysis, the dialysis tube was weighed again to estimate the dilution factor of the medium (C_{trastuzumab}=88 g/L; C_{MEX-CD2}=52 g/L).

The pH and osmolarity of the mixture were measured using a Mettler Toledo SevenCompact S210 pH meter and a Camlab Löser Micro MOD200 Plus osmometer respectively (pH=5.6; osmolarity=268 mOsm/L).

Injection tests with different needle diameters and a 1 mL syringe were performed to qualitatively assess the injection capacity of the solution. A score from 1 to 10 was given to the ability to inject through the needle, 1 being the score given to a solution which is not or very difficultly injectable; 10 being the score given to a very easy injection.

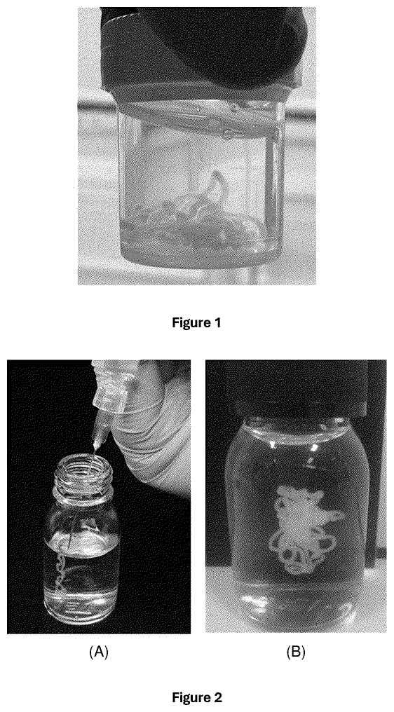

Solution gelation tests were also performed in a buffered hemodialysis solution (Hemosol B0, an hemodialysis/hemofiltration solution comprising 1.75 mmol/L of Ca²⁺, 0.5 mmol/L Mg²⁺, 140 mmol/L of Cl⁻, 3 mmol/L of lactate and 32 mmol/L of HCO₃⁻) to qualitatively assess the strength of the hydrogel formed. A score from 1 to 10 is given to the gel's resistance in the medium, 10 being the score given to a very resistant gel. The results are presented in Table 1 and an example of hydrogel obtained is illustrated in FIG. 1.

TABLE 1
Qualitative estimation of the injection and gelation capacities
of the MEX-CD2-I-tmb solution synthesized according to
method 1 and with different needle diameters

| Needle diameter | | | Gelation score in |
|---|---|---|---|
| (mm) | Gauge | Injection score | Hemosol B0 |
| 0.70 | 22 | 10 | 7 |
| 0.60 | 23 | 9 | 6 |
| 0.50 | 25 | 7 | 5 |
| 0.40 | 27 | 5 | 4 |

Table 1 - Qualitative estimation of the injection and gelation capacities of the MEX-CD2-I-tmb solution synthesized according to method 1 and with different needle diameters

These results show that the synthesized solution is easily injectable by an operator and concerns a wide range of needles commonly used in the medical field for the subcutaneous injection of active substances or various pharmaceutical products. Moreover, an in situgelation of the solution was observed after injection in a medium with physiological conditions (pH=7.4 and osmolarity=300 mOsm/L).

### Example 2: Preparation of a Formulation MEX-CD2-I-tmb (Also Referred to as MEX-CD2-I-HER2) in Accordance with the Invention, According to a Method 2

According to a method (2), the preparation of MEX-CD2-I-tmb was carried out from a viscous solution of composition referred to as MEX-CD2-I solution with a ratio α/β % where α is the mass concentration (w/w) of chitosan A and β the mass concentration (w/w) of polysaccharide B in the considered formulation.

Briefly, 1.33 g of polysaccharide B (MEX-CD2) and 0.67 g of chitosan A were dispersed in 19.87 mL of milli-Q water and 150 μL of ultrapure acetic acid was added with mechanical stirring at 100 rpm in a 50 mL reactor. The mixture was left to stir for 2 hours until complete dissolution and homogenization of the medium. The resulting solution was recovered and introduced into a suitable fluid dispenser, then centrifuged at 4000 rpm for 10 minutes to obtain an air bubble-free solution comprising the composition. The pH and osmolarity of the mixture were measured using a Mettler Toledo SevenCompact S210 pH meter and a Löser Micro MOD200 Plus osmometer from Camlab respectively (pH=5.9; osmolarity=503 mOsm/L).

According to this method (2), two vials of Trazimera®420 mg are reconstituted at 50 g/L in 8.4 mL of milli-Q water and the trastuzumab solution was homogenized using a vortex shaker at 1000 rpm for 5 minutes. The trastuzumab solution was then introduced into a Sartorius Vivaspin™ 20 centrifugal concentrator (polyethersulfone membranes; cut-off: 100 kDa) and centrifuged for 4 h at 4000 rpm. This allows to separate the trastuzumab antibody from the excipients with whom it is formulated in the Trazimera® commercial solution. The retentate was collected and the concentration of trastuzumab was monitored by measuring the absorbance at 275 nm using a Varian Cary® 50 UV-Vis spectrophotometer. The trastuzumab concentration was readjusted to 200 g/L by adding milli-Q water and the pH and osmolarity of the trastuzumab solution were measured (pH=6.19; osmolarity=129 mOsm/L).

In one method (2a), 2 mL of the trastuzumab solution was introduced into a Luer Lock syringe and 2 mL of the 3.3/6.7% solution was introduced into another Luer Lock syringe. The two syringes were connected using a Luer Lock connector and were thoroughly mixed by repeated injections of the contents of one solution into the other and vice versa (approximately 200 times). The pH and osmolarity of the solution are measured (pH=5.77; osmolarity=228 mOsm/L).

In another method (2b), 2 mL of the trastuzumab solution and 2 mL of the 3.3/6.7% solution were thoroughly mixed in a reactor by mechanical agitation using a rotor having blades at 100 rpm for 2 hours. The pH and osmolarity of the solution are measured (pH=5.77; osmolarity=243 mOsm/L).

Injection tests with different needle diameters and a 1 mL syringe were performed to qualitatively assess the injection capacity of the solution. A score from 1 to 10 was given with respect to the ability to inject through the needle, 10 being the score given to a very easy injection. Solution gelation tests were also performed in physiological serum solution to qualitatively assess the strength of the hydrogel formed. A score from 1 to 10 was given for the gel's resistance in the medium, with 10 being the score for a very resistant gel. The results are presented in Table 2.

TABLE 2
Qualitative estimation of injection and gelation
capacities with different needle diameters

| | | | |
|---|---|---|---|
| Needle diameter | | | Gelation score in |
| (mm) | Gauge | Injection score | NaCl 9 g/L |

MEX-CD2-I-tmb solution synthesized
according to method 2a

| | | | |
|---|---|---|---|
| 0.90 | 20 | 10 | 10 |
| 0.70 | 22 | 10 | 10 |
| 0.60 | 23 | 9 | 9 |
| 0.50 | 25 | 7 | 8 |
| 0.40 | 27 | 4 | 8 |

MEX-CD2-I-tmb solution synthesized
according to method 2b

| | | | |
|---|---|---|---|
| 0.90 | 20 | 10 | 8 |
| 0.70 | 22 | 10 | 8 |
| 0.60 | 23 | 9 | 7 |
| 0.50 | 25 | 8 | 6 |
| 0.40 | 27 | 5 | 5 |

Table 2 - Qualitative estimation of injection and gelation capacities with different needle diameters

These results demonstrate the possibility of formulating a solution that is easily injectable by an operator and capable of gelling in situ under physiological conditions even after purification of the excipients initially contained in a commercial solution of trastuzumab (Trazimera®). This study also demonstrates that it is possible to mix the antibody with the polymer solution in different ways without affecting the injection and gelling capabilities of the final solution.

### Example 3: Preparation of a Formulation MEX-CD2-I-tmb (Also Referred to as MEX-CD2-I-HER2) in Accordance with the Invention, with Fluorescent Marking

According to a method (3), polysaccharide B was chemically pre-labelled with cyanine 5.5 (Cy5.5). According to this method, trastuzumab is also chemically labelled with fluorescein by grafting with fluorescein isothyocyanate (FITC).

The labelling of polysaccharide B with cyanine 5.5 was achieved by direct addition of the fluorophore into a modified chitosan solution. During the synthesis and purification steps, the solution was protected from light by means of aluminium foil. Briefly, a cyanine 5.5 solution at 5 g/L was made by dissolving 100 mg of cyanine 5.5 in 20 mL of anhydrous DMSO.

In parallel, a solution of polysaccharide B was made by complete dissolution of 1.1 g of polysaccharide B in 100 mL of milli-Q water. The pH of this solution was adjusted to pH=6.02 by adding 750 μL of a 1 mol/L NaOH solution under stirring. The cyanine 5.5 solution was added dropwise to the polysaccharide B solution with stirring at 200 rpm, so that one free NH₂ function of polysaccharide B out of 27 was grafted with a cyanine 5.5 molecule (molar ratio=0.037). The reaction mixture was left for 24 hours under stirring at 100 rpm in the dark. The synthesis product was then purified by tangential filtration using the Sartoflow® Smart device with a Sartocon® Slice PESU cassette (polyethersulfone membranes; cut-off: 100 kDa; filtration area: 200 cm²) according to a diafiltration-concentration model against 10 L of a 5 mM acetic acid solution. The synthesis product was then lyophilized for 48 h.

The labelling of trastuzumab with fluorescein (sample 3a) was also carried out by direct addition of the fluorophore into a solution of Trazimera®, from which the excipients have been previously removed (compound 3b). First, two vials of Trazimera® 420 mg were reconstituted at 50 g/L in 8.4 mL of milli-Q water and the solution was left to stand at 4° C. for 24 h. The solution was then introduced into a Sartorius Vivaspin™ 20 centrifugal concentrator (polyethersulfone membranes; cut-off: 100 kDa) and centrifuged for 7 h at 4000 rpm. The retentate was collected and the concentration of trastuzumab monitored by measuring the absorbance at 275 nm using a Varian Cary® 50 UV-Vis spectrophotometer. The trastuzumab concentration was measured at 247 g/L.

A 5 g/L trastuzumab solution was then made by diluting 50 μL of this purified trastuzumab solution (compound 3b) in 1.95 mL of carbonate-bicarbonate buffer at pH=9.06. In parallel, 1.9 mg of FITC was dissolved in 1.9 mL of anhydrous DMSO. The FITC solution was then added dropwise to the purified trastuzumab solution under agitation of 200 rpm, such that one molecule of trastuzumab was grafted with 3.7 molecules of fluorescein. The reaction mixture was left for 3 hours under agitation at 200 rpm in the dark. The resulting solution was then introduced into a SpectraPor® Float-A-Lyzer® dialysis tube (cut-off: 5 kDa) and dialysed for 18 hours against 800 mL of 0.1 M phosphate buffer solution at pH=7.4. The solution obtained was finally dialysed for 2 hours against 800 mL of milli-Q water.

According to this method, a viscous solution referred to as composition 3.3/6.7% with a ratio α/β % where α is the mass concentration (w/w) of chitosan A and β the mass concentration (w/w) of cyanine 5.5 labelled polysaccharide B was made (compound 3c). Briefly, 667 mg of cyanine 5.5 labelled polysaccharide B (MEX-CD2) and 333 mg of chitosan A are dispersed in 8.92 mL of milli-Q water and 75 μL of ultrapure acetic acid was added with mechanical stirring at 100 rpm in a 50 mL reactor. The mixture was left to stir for 2 hours until complete dissolution and homogenisation of the medium. The solution obtained was recovered and introduced into a suitable fluid dispenser, then centrifuged at 4000 rpm for 10 minutes to obtain a solution without air bubbles comprising the composition. In parallel, a 3.3/6.7% solution identical to that described in Example 2 was synthesized (compound 3d).

The fluorescently labelled MEX-CD2-I-tmb solution was finally prepared as follows: first, 0.9 mL of the 3.3/6.7% mixture based on unlabelled polysaccharide B (compound 3d) was introduced into a Luer Lock syringe and 0.1 mL of the 3.3/6.7% mixture made from cyanine 5.5-labelled polysaccharide B was introduced into another Luer Lock syringe. The two syringes were connected with a Luer Lock connector and were thoroughly mixed by repeated injections of the contents of one into the other and vice versa (compound 3e). In parallel, 0.2 mL of the 5 g/L fluorescein-labelled trastuzumab solution (compound 3a) was added with agitation at 100 rpm to 0.8 mL of purified 247 g/L trastuzumab (compound 3b). The resulting solution was recovered and inserted into a suitable Luer Lock syringe (compound 3f). The syringe containing compound 3e and the one containing compound 3f were finally connected with a Luer Lock connector and thoroughly mixed by repeated injections of the contents of one into the other and vice versa (100 to 200 times). The pH and osmolarity of the mixture were measured using a Mettler Toledo SevenCompact S210 pH meter and a Camlab Löser Micro MOD200 Plus osmometer respectively (pH=5.69; osmolarity=285 mOsm/L).

A gelation and injection test of this solution was carried out with a 1 mL syringe equipped with a 22 G needle by direct injection into a 0.9% (w/w) NaCl solution and into a 0.1M PBS (phosphate buffer saline) solution. The solution was easily injected by the operator and gelled instantly on contact with physiological serum or PBS. In addition, the gel was qualitatively much more resistant in PBS than in physiological serum.

The in situ gelation of MEX-CD2-I-tmb solution with fluorescent marking is illustrated in Figure (2A), the gel obtained after complete gelation is illustrated in Figure (2B).

This study shows the possibility of easily and independently labelling the polymer or antibody with different fluorophore probes without drastically altering the physicochemical properties of the resulting solutions. The injection and gelation capabilities of the resulting solutions are also comparable to solutions obtained without prior labelling of the reagents.

### Example 4: Study of the Rheological Properties of MEX-CD2-I Solutions (Before Mixing with the Antibody) as a Function of pH and Osmolarity (not According to the Invention)

The aim of this study was to determine the physico-chemical parameters of the precursor polymer solution (before mixing with the antibody) for which the compound remains stable in solution and does not cause significant gelation of the system. Therefore, solutions of MEX-CD2-I at 5% (w/w) were prepared at different osmolarity and pH, and their rheological properties were measured to establish the range of osmolarity and pH that lead to gelation of the solution.

For the preparation of the samples with osmolarity variation, a viscous solution of composition referred to as 1.7/3.3% with a ratio α/β % where α is the mass concentration (w/w) of chitosan A and β the mass concentration (w/w) of polysaccharide B was made.

Briefly, 1.67 g of polysaccharide B (MEX-CD2) and 0.83 g of chitosan A are dispersed in 47.33 mL of milli-Q water and 162 μL of ultrapure acetic acid was added under mechanical stirring at 100 rpm in a 50 mL reactor. The mixture was left to stir for 2 hours until complete dissolution and homogenisation of the medium. The resulting solution was collected and introduced into a suitable fluid dispenser, then centrifuged at 4000 rpm for 10 minutes to obtain an air bubble-free solution comprising the composition. The pH and osmolarity of the mixture are measured using a Mettler Toledo SevenCompact S210 pH meter and a Löser Micro MOD200 Plus osmometer from Camlab respectively (pH=5.75; osmolarity=57 mOsm/L). 2 mL of this viscous solution was introduced into an adapted Luer Lock syringe and a mass of NaCl was introduced into another Luer Lock syringe. The contents of the two syringes are thoroughly mixed by repeated injections of the contents of one into the other and vice versa using a Luer Lock connector to obtain samples with an osmolarity ranging from 57 mOsm/L to 352 mOsm/L at an identical pH of 5.75.

For the preparation of the samples with pH variation, the samples were obtained from the same 1.7/3.3% solution as described above.

2 ml of this solution was added to a SpectraPor® Float-A-Lyzer® dialysis tube (cut-off: 50 kDa). Prior to dialysis, the dialysis devices were washed in a 10% (V/V) ethanol solution for 30 minutes and then washed in milli-Q water for 30 minutes.

Dialysis was then conducted against an isotonic NaCl solution (1.9 g/L NaCl which is equivalent to an osmolarity of 57 mOsm/L) in order to maintain a stable concentration of the solution in the dialysis membrane throughout the experiment.

After 3 hours, the pH of the dialyzed solution was adjusted to pH=6.0 by addition of NaOH and the solution left for a period ranging from 18 to 72 hours depending on the desired pH of the sample. Samples with an osmolarity ranging from 55 to 98 mOsm/L and a pH ranging from 5.75 to 6.4 were obtained.

The rheological properties of each sample were determined using a TA Instruments AR2000 rheometer. Specifically, measurements were made using a 4° and 25 mm planar cone geometry. Prior to the measurement, the zero deviation was set and the inertia and rotation mapping of the instrument was calibrated. The samples are then spread out on the stage and the deviation from the geometry was set to 116 microns. The excess sample was removed with a spatula to limit edge effects.

A preliminary study was carried out to determine the limit of the linear deformation domain of the materials. To do this, a 10 Hz amplitude sweep was performed by varying the amplitude from 0.1% to 100% with an operating temperature of 25LC.

First, the storage modulus G′ and loss modulus G″ of each sample were determined at 25° C. by sweeping the oscillation frequency at a fixed strain of 10% (6.9.105 rad) for angular frequencies ranging from 1 to 100 rad·s⁻¹. G′ and G″ thus define the viscoelastic behavior of solutions and gels. Then, the viscosity of each sample was measured without removing the sample from the tray with a flow sweep study conducted by sweeping the shear rate from 10⁻² to 10² s⁻¹ at an operating temperature of 25° C. The storage modulus G′ characterizes the elasticity of the material and increases during the gelation state of the system and is characterizing flow behavior of solutions. The loss modulus G″ characterizes the viscous behavior of the system. The results obtained on each sample are presented in Table 3. The materials exhibit a solution state (G″>G′) or a soft gel state (when G′>G′).

TABLE 3
Physico-chemical and rheological properties for each sample

| | | | | |
|---|---|---|---|---|
| | | Newtonian | Storage modulus | Loss modulus |
| | Osmolarity | viscosity | G′ (Pa), | G″ (Pa), |
| pH | (mOsm/L) | (Pa · s) | (at 0.15 Hz) | (at 0.15 Hz) |

Samples obtained by variation of osmolarity

| | | | | |
|---|---|---|---|---|
| 5.75 | 57 | 15.9 | 0.26 | 2.27 |
| 5.78 | 137 | 19.4 | 0.57 | 2.56 |
| 5.76 | 161 | 19.0 | 0.42 | 2.60 |
| 5.69 | 197 | 24.5 | 0.80 | 2.74 |
| 5.76 | 239 | 20.5 | 1.25 | 2.10 |
| 5.75 | 285 | 67.2 | 6.02 | 6.13 |
| 5.82 | 352 | 399.1 | 26.25 | 20.76 |

Samples obtained by variation of pH

| | | | | |
|---|---|---|---|---|
| 5.75 | 57 | 15.9 | 0.26 | 2.27 |
| 6.0 | 98 | 75.8 | 0.92 | 3.13 |
| 6.28 | 77 | 68.8 | 2.74 | 6.76 |
| 6.37 | 55 | 23.4 | 0.11 | 0.21 |

Table 3 - Physico-chemical and rheological properties for each sample

The storage modulus G′ is lower than the loss modulus G″ for the sample with an osmolarity below 352 mOsm/L. Therefore, above 350 mOsm/L, the system tends to be in a gelled state. The gelation point can be determined when the moduli G′ and G″ are equal. At a pH of 5.75, the gelation point appears to occur at an osmolarity of around 300 mOsm/L. Therefore, gelation of the system at pH=5.75 occurs at an osmolarity above 300 mOsm/L.

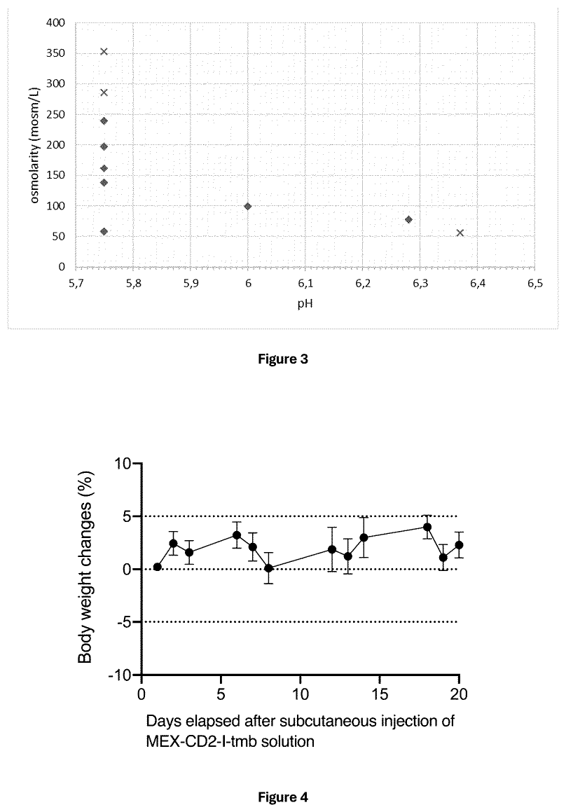

The stability ranges of MEX-CD2-I solutions assayed as a function of pH and osmolarity is summarized in FIG. 3.

The results obtained on the samples with varying pH show heterogeneity in rheological properties due to the heterogeneity of the medium caused by the dialysis method used. However, for an osmolarity ranging from 50 to 100 mOsm/L, a gelation of the system was qualitatively observed when the pH was higher than 6.4.

### Example 5: Study of the Physicochemical Properties of MEX-CD2-I-tmb (Also Referred to as MEX-CD2-HER2) Solution as a Function of Trastuzumab Concentration

The aim of this study was to determine the optimal concentration of trastuzumab in the MEX-CD2-I-tmb solution for which the compound has physicochemical properties suitable for subcutaneous injection of the product. Samples of MEX-CD2-I-HER2 comprising 5% (w/w) polymer were prepared at different concentrations of commercial trastuzumab solution purified and unpurified from its excipients and ranging from 12.5 g/L to 75 g/L. For each sample, the pH and osmolarity of the solution were measured using a Mettler Toledo SevenCompact S210 pH meter and a Camlab Löser Micro MOD200 Plus osmometer respectively.

Briefly, the samples were made from a viscous solution of composition 3.3/6.7% identical to that described in Example 2 (sample 5a). In parallel, a vial of Trazimera® 420 mg was reconstituted to 100 g/L in 4.2 mL of milli-Q water and the solution was homogenized using a vortex mixer at 1000 rpm for 5 minutes. This solution is referred to as the trastuzumab solution with excipients (sample 5b). Two further vials of Trazimera® 420 mg are reconstituted to 50 g/L in 8.4 mL of milli-Q water and the solution was homogenized using a vortex mixer at 1000 rpm for 5 minutes. The solution was then introduced into a Sartorius Vivaspin™ 20 centrifugal concentrator (polyethersulfone membranes; cut-off: 100 kDa) and was purified using the same protocol described in Example 2. The resulting solution was diluted to 100 g/L with milli-Q water and is referred to as trastuzumab solution without excipients (compound 5c).

MEX-CD2-I-tmb samples were made by introducing an appropriate volume of 3.3/6.7% solution into a 2 mL Luer Lock syringe. In parallel, different volumes of trastuzumab solution (purified or not) were introduced into a 2 mL Luer Lock syringe in order to obtain final MEX-CD2-I-tmb solutions with trastuzumab concentrations ranging from 12.5 g/L to 75 g/L. The two syringes are connected with a Luer Lock connector and were thoroughly mixed by repeated injections of the contents of one into the other and vice versa (approximately 200 times). The pH and osmolarity of the solution were then measured.

For example, the synthesis of the 50 g/L sample of trastuzumab unpurified from the excipients was performed as follows: 1 mL of 3.3/6.7% solution was introduced into a 2 mL Luer Lock syringe. 1 mL of unpurified trastuzumab solution (compound 5b) was introduced into a 2 mL Luer Lock syringe. The two syringes were connected with a Luer Lock connector and were thoroughly mixed manually by repeated injection of the contents of one into the other and vice versa (approximately 200 times).

The results obtained on each sample are presented in Table 4.

TABLE 4
Physico-chemical properties of MEX-CD2-I-tmb solutions obtained
from trastuzumab solution purified or unpurified of excipients
as a function of trastuzumab concentration.

| | | | |
|---|---|---|---|
| Polymer | Trastuzumab | | Osmolarity |
| concentration (g/kg) | concentration (g/L) | pH | (mOsm/L) |

Samples obtained from trastuzumab solution
purified from the excipients (5c)

| | | | |
|---|---|---|---|
| 50 | 0 | 5.77 | 85 |
| | 12.5 | 5.82 | 101 |
| | 25 | 5.83 | 152 |
| | 37.5 | 5.85 | 124 |
| | 50 | 5.87 | 106 |
| | 75 | 5.88 | 226 |

Samples obtained from trastuzumab solution
unpurified from the excipients (5b)

| | | | |
|---|---|---|---|
| 50 | 12.5 | 5.7 | 264 |
| | 25 | 5.73 | 262 |
| | 37.5 | 5.68 | 212 |
| | 75 | 5.68 | 312 |

Table 4 - Physico-chemical properties of MEX-CD2-I-tmb solutions obtained from trastuzumab solution purified or unpurified of excipients as a function of trastuzumab concentration.

This study shows the biocompatibility of the MEX-CD2-I-tmb solution for subcutaneous injections as a function of trastuzumab concentration. For samples obtained from the purified solution of the excipients, the pH increases very slightly as a function of trastuzumab concentration. This increase is due to the fact that the purified trastuzumab solution has a higher pH than the 3.3/6.7% solution (pH=6.19). There is also an increase in osmolarity as a function of trastuzumab concentration due to the contribution of osmotically active species contained in the trastuzumab solution. This trend is even more significant for samples obtained from unpurified trastuzumab solution.

### Example 6—Study of the Gelling Capacity of the Solution as a Function of the MEX-CD2/Chitosan a Ratio (not According to the Invention)

The aim of this study was to determine the gelation capacity of the MEX-CD2-I solution as a function of the starting MEX-CD2/Chitosan A weight ratio. Respectively, solutions with ratios 3.5/0%; 0/7% and 2.5/2.5% were made according to a ratio α/β % where α is the mass concentration (w/w) of chitosan A and β the mass concentration (w/w) of polysaccharide B. For each sample, the pH and osmolarity of the solution were measured using a Mettler Toledo SevenCompact S210 pH meter and a Löser Micro MOD200 Plus osmometer from Camlab respectively.

Briefly, the 3.5/0% solution was made by dissolving 350 mg of chitosan A in 10 mL of milli-Q water in a 50 mL reactor. Glacial acetic acid was added under mechanical stirring until a clear mixture was obtained where all the chitosan was solubilized (100 μL). The mixture was left under stirring for 2 hours until complete dissolution and homogenization of the medium. The solution obtained was recovered and introduced into a suitable fluid dispenser (pH=5.64; osmolarity=101 mOsm/L).

The 0/7% solution was made by dissolving 700 mg of MEX-CD2 in 10 mL of milli-Q water in a 50 mL reactor. Glacial acetic acid was added under mechanical stirring until a clear mixture was obtained where all the chitosan was solubilized (50 μL). The mixture was left under stirring for 2 hours until complete dissolution and homogenization of the medium. The solution obtained was recovered and introduced into a suitable fluid dispenser (pH=4.79; osmolarity=157 mOsm/L).

The 2.5/2.5% solution was made by dissolving 250 mg of chitosan A and 250 mg of MEX-CD2 in 10 mL of milli-Q water in a 50 mL reactor. Glacial acetic acid was added under mechanical stirring until a clear mixture was obtained where the entire amount of polymer was solubilized (80 μL). The mixture was left to stir for 2 hours until complete dissolution and homogenization of the medium. The solution obtained was recovered and introduced into a suitable fluid dispenser (pH=5.18; osmolarity=117 mOsm/L).

300 μL of each solution was injected using a 1 mL syringe and a 25 G needle into 20 mL of physiological serum solution (NaCl 9 g/l) or 20 mL of commercial phosphate buffer saline (PBS) at 10 mmol/L. For each injection test, the gelation of the solution was estimated qualitatively after 30 minutes. A score of 1 to 10 was assigned to the ability to inject through the 25 G needle, with 10 being the score for very easy injection. A score of 1 to 10 was also given for the gel's resistance in the medium, with 10 being the score for a very resistant gel. The results are presented in Table 5.

TABLE 5
Qualitative estimation of the injection and gelation capacities of
the MEX-CD2-I solution according to the α/β % ratio

| | | | | Gelation | |
|---|---|---|---|---|---|
| Weight Ratio | | | | score in | Gelation |
| (Chitosan A/ | | Osmolarity | Injection | physiological | score in |
| MEX-CD2) | pH | (mOsm/L) | Score | serum | PBS |
| 3.5/0% | 5.64 | 101 | 8 | 0 | 3 |
| 0/7% | 4.79 | 157 | 9 | 7 | 9 |
| 2.5/2.5% | 5.18 | 117 | 6 | 4 | 7 |

Table 5 - Qualitative estimation of the injection and gelation capacities of the MEX-CD2-I solution according to the α/β % ratio.

The gelling of the solution in physiological serum solution is mainly due to the presence of MEX-CD2 in the solution. In PBS, chitosan A tends to precipitate and form a very soft gel with little cohesion but still contributes to the gelation of the system. A solution with a high proportion of MEX-CD2 will have a greater tendency to gel easily while chitosan is added to obtain zones of crystallinity as described in a previous patent application FR2110474.

### Example 7: Preparation of a Formulation MEX-CD2-I-HER2 (Also Referred to as MEX-CD2-tmb) in Accordance with the Invention, According to a Method (4)

### 7.1. General Preparation Procedure of MEX-CD2-I-HER2 According to a Method (4)

According to a method (4), the preparation of MEX-CD2-I-HER2 was carried out from a viscous solution of composition referred to as a 3.3/6.7% MEX-CD2-I solution with a ratio α/β % where α is the mass concentration (w/w) of chitosan A and β the mass concentration (w/w) of polysaccharide B in the considered formulation.

Briefly, 667 mg of polysaccharide B (MEX-CD2) and 133 mg of chitosan A were dispersed in 8.92 mL of milli-Q water and 72 μL of ultrapure acetic acid was added with mechanical stirring at 100 rpm in a 50 mL reactor. The mixture was left to stir for 2 hours until complete dissolution and homogenization of the medium. The resulting solution was recovered and introduced into a suitable fluid dispenser, then centrifuged at 4000 rpm for 10 minutes at room temperature to obtain an air bubble-free solution comprising the composition. The mixture is then introduced into glass syringes and sterilized for 20 min at 121° C. in an autoclave. The pH and osmolarity of the mixture were measured using a Mettler Toledo SevenCompact S210 pH meter and a Löser Micro MOD200 Plus osmometer from Camlab respectively (pH=5.7; osmolarity=410 mOsm/L). Newtonian viscosity of the resulting solution was measured with a Thermo Scientific HAAKE RheoStress 600 Sensor Systems using a C35/2° Ti L cone plate geometry. Viscosity was measured with a flow sweep linear study conducted by scanning shear rate from 10⁻² to 10³ s⁻¹ at a regulated temperature of 25° C. 10 points per decade are recorded with a measurement time of 10 seconds for each value. Newtonian viscosities of η₀=2023 Pa·s and η₀=3260 Pa·s were obtained for the solution before and after sterilization respectively.

According to this method (4), a vial of Trazimera® 420 mg is reconstituted at 200 g/L in 2.1 mL of milli-Q water and the trastuzumab solution is left at 4° C. for 24 h until complete dissolution and homogenization is obtained.

According to this method (4), 1.2 mL of the trastuzumab solution was introduced into a Luer Lock syringe and 1.2 mL of the 3.3/6.7% solution was introduced into another Luer Lock syringe. The two syringes were connected using a Luer Lock connector and were thoroughly mixed by repeated injections of the contents of one solution into the other and vice versa (approximately 50 to 200 times). The pH, osmolarity and Newtonian viscosity of the solution are measured (pH=5.81; osmolarity=564 mOsm/L; η₀=126.7 Pa·s). The injectability of this MEX-CD2-I-HER2 solution was precisely measured using a Shimadzu AG-X plus force machine. The solution was introduced into a BD Hylok™ 1 mL pre-fillable glass syringe fitted with a Terumo Agani 25 G needle (0.5×16 mm). Injectability was determined as the force in Newton required to eject the solution at a constant plunger velocity of 1 mm/s. Ejection force of Fₑ=46.7 N is obtained meaning that the system can be manually injected by a practitioner (T. E. Robinson et al., Filling the Gap: A Correlation between Objective and Subjective Measures of Injectability, Adv. Healthc. Mater., vol. 9, no 5, p. 1901521, 2020).

A gelation test of this solution was carried out with a 1 mL syringe equipped with a 25 G needle by direct injection into a 10 mM PBS (phosphate buffered saline) solution. The solution gelled instantly on contact with PBS.

7.2. Preparation Procedure for MEX-CD2-I-HER2 Labeled with Gadolinium Marking

According to a method described below, polysaccharide B was chemically labelled with gadolinium by complexation of gadolinium Gd³⁺ ions on its pre-grafted chelating groups.

The labelling of polysaccharide B with gadolinium was achieved by direct addition of a 1 mol/L solution of gadolinium into a modified chitosan solution. Briefly, 8.74 mL of a 1 mol/L solution of Gd³⁺ were added to 1 L of a 7 g/L solution of polysaccharide B under stirring. The pH is then adjusted from pH=3.6 to pH=5.6 by addition of a 1 mol/L sodium hydroxide solution. The solution was left under agitation at 60° C. for 48 hours. The synthesis product was then purified by tangential filtration using the Sartoflow® Smart device with a Sartocon® Slice PESU cassette (polyethersulfone membranes; cut-off: 100 kDa; filtration area: 200 cm²) according to a diafiltration-concentration model against 10 L of a 5 mM acetic acid solution. The synthesis product was then lyophilized for 48 h. The amount of gadolinium complexed onto the grafted chelating group of polysaccharide B was measured by ICP-MS ¹⁵⁸Gd analysis conducted on a PerkinElmer NexION 2000 and was estimated to be 70% of the total DOTAGA groups complexed with gadolinium.

According to this method, a viscous solution referred to as composition 3.3/6.7% with a ratio α/β % where α is the mass concentration (w/w) of chitosan A and β the mass concentration (w/w) of total polysaccharide B (comprising unmodified compound and Gd labelled compound) was made. Briefly, 601.9 mg of polysaccharide B, 66.2 mg of gadolinium labelled polysaccharide B and 332.9 mg of chitosan A are dispersed in 8.92 mL of milli-Q water and 76 μL of ultrapure acetic acid was added with mechanical stirring at 100 rpm in a 50 mL reactor. The mixture was left to stir for 2 hours until complete dissolution and homogenisation of the medium. The solution obtained was recovered and introduced into a suitable fluid dispenser, then centrifuged at 4000 rpm for 10 minutes to obtain a solution without air bubbles comprising the composition. The mixture is then introduced into glass syringes and sterilized for 20 min at 121° C. in an autoclave.

According to this method, a vial of Trazimera® 420 mg is reconstituted at 200 g/L in 2.1 mL of milli-Q water and the trastuzumab solution is left at 4° C. for 24 h until complete dissolution and homogenization is obtained.

According to this method, 1 mL of the trastuzumab solution was introduced into a Luer Lock syringe and 1 mL of the 3.3/6.7% solution was introduced into another Luer Lock syringe. The two syringes were connected using a Luer Lock connector and were thoroughly mixed by repeated injections of the contents of one solution into the other and vice versa (approximately 50 to 200 times).

7.3. Preparation Procedure for MEX-CD2-I-HER2 Labeled with Fluorescent Marking and Gadolinium Labelling

According to a method described below, polysaccharide B was chemically pre-labelled with cyanine 5.5 (Cy5.5). According to this method, polysaccharide B is also chemically labelled with gadolinium by complexation of gadolinium Gd³⁺ ions on its pre-grafted chelating groups.

The labelling of polysaccharide B with cyanine 5.5 was achieved using the same procedure as described in Example 3.

The labelling of polysaccharide B with gadolinium was achieved using the same procedure as described in Example 7.2.

According to this method, a viscous solution referred to as composition 3.3/6.7% with a ratio α/β % where α is the mass concentration (w/w) of chitosan A and β the mass concentration (w/w) of total polysaccharide B (comprising unmodified compound, Cy5.5 labelled compound and Gd labelled compound) was made. Briefly, 318 mg of polysaccharide B, 7.63 mg of cyanine 5.5 labelled polysaccharide B, 17.2 mg of gadolinium labelled polysaccharide B and 167.5 mg of chitosan A are dispersed in 4.46 mL of milli-Q water and 38 μL of ultrapure acetic acid was added with mechanical stirring at 100 rpm in a 50 mL reactor. The mixture was left to stir for 2 hours until complete dissolution and homogenisation of the medium. The solution obtained was recovered and introduced into a suitable fluid dispenser, then centrifuged at 4000 rpm for 10 minutes to obtain a solution without air bubbles comprising the composition. The mixture is then introduced into glass syringes and sterilized for 20 min at 121° C. in an autoclave.

According to this method, a vial of Trazimera® 420 mg is reconstituted at 200 g/L in 2.1 mL of milli-Q water and the trastuzumab solution is left at 4° C. for 24 h until complete dissolution and homogenization is obtained.

According to this method, 0.5 mL of the trastuzumab solution was introduced into a Luer Lock syringe and 0.5 mL of the 3.3/6.7% solution was introduced into another Luer Lock syringe. The two syringes were connected using a Luer Lock connector and were thoroughly mixed by repeated injections of the contents of one solution into the other and vice versa (approximately 50 to 200 times).

### Example 8: In Vivo Study of a MEX-CD2-I-tmb (Also Referred to as MEX-CD2-I-HER2 Solutions in Accordance with the Invention

Unless stated otherwise, the MEX-CD2-I-tmb solution used in this Example was prepared according to Example 1.

### 8.1. In Vivo Toxicity Study

16 mice were injected subcutaneously (at the neck) with 200 μL of MEX-CD2-I solution (not according to the invention) and 9 mice with 250 μL of a MEX-CD2-I-tmb solution loaded with 100 g/L of trastuzumab antibody (corresponding to a dose of 15 mg anti-HER2 antibody) prepared according to Example 1.

Weight monitoring of the mice was performed. The results show that no gross toxicity occurred in the mice. The weight measurement after subcutaneous injection of 200 μL of MEX-CD2-I-HER2 solution loaded with 100 g/L of trastuzumab antibody are shown in FIG. 4.

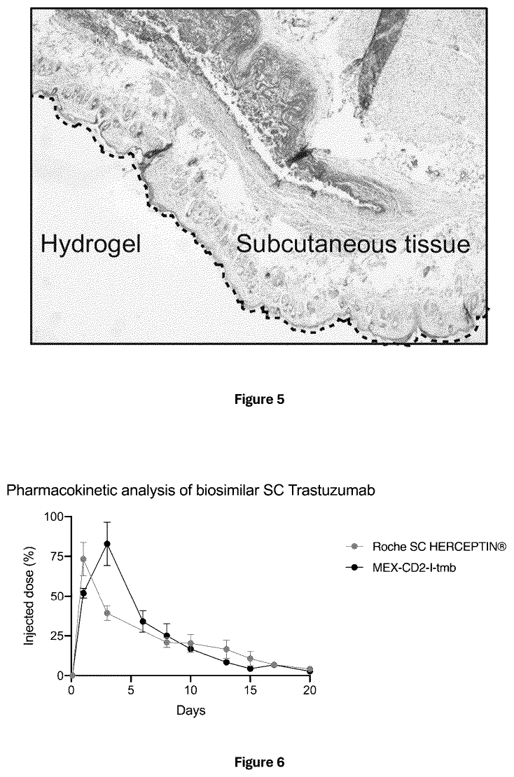

A hematoxylin and eosin (H&E) immunohistochemical study was performed on 3 mice 20 days after injection. The results are illustrated in FIG. 5. No cell recruitment (macrophages) was observed, confirming the absence of inflammation.

### 8.2. In Vivo Study of the Sustained Release of the Antibody by the Hydrogel

For this study, we compared the presence in the plasma of anti-HER2 antibodies (trastuzumab) following its release from the hydrogel that formed in situ after subcutaneous injection of 200 μL of a MEX-CD2-I-tmb solutions loaded with 100 g/L of trastuzumab antibody (corresponding to a dose of 15 mg of trastuzumab antibody) over 20 days by HTRF (Homogeneous Time Resolved Fluorescence) quantification to the controlled release of the same antibody after subcutaneous injection of 200 μL of HERCEPTIN® SC formulation (corresponding to dose of trastuzumab antibody of 15 mg per injection).

9 mice per group were used for this study with 1 blood sample every 3 days from 3 mice. The results are shown in FIG. 6.

The results show a time shift of the maximum presence (Cₘₐₓ) from 1 day (HERCEPTIN® SC formulation) to 3 days with our MEX-CD2-I-tmb solution. Further, at the end of this experiment (Day 26 after SC injection), no trace of hydrogel was present at the injection site, confirming the biodegradability of the formulation.

### 8.3. In Vivo Study of the Biodegradability of the Hydrogel

The polymer was functionalized with a Cyanine 5.5 fluorescent probe to follow its biodegradation after subcutaneous injection. 200 μL of such fluorescently labelled MEX-CD2-I-tmb prepared according to Example 3 was injected into 3 mice and longitudinal monitoring was performed every 3 days by whole body fluorescence imaging (IVIS, PerkinElmer).

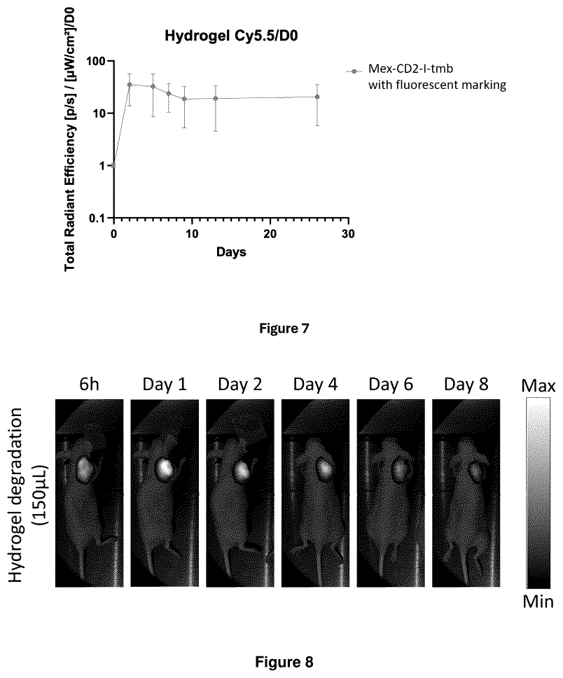

The results are shown in FIG. 7.

For this formulation of Cyanine 5.5 labelled hydrogel, we observe a stable signal plateau up to 26 days after implantation confirming the possibility of modifying the hydrogel formulation to extend its subcutaneous lifetime.

### Example 9: In Vivo Biodegradability and Toxicity Study of a MEX-CD2-I-HER2 Solutions in Accordance with the Invention

### 9.1. Study of In Vivo Biodegradability by Fluorescence

Solutions of MEX-CD2-I-HER2 were prepared according to the process of Example 7.3.

Part of the MEX-CD2 used for the formulation was labelled with a Cyanine 5.5 (Cy5.5) fluorescent dye, and another part was complexed with Gadolinium ions Gd3⁺. 150 μL of the final solution loaded with 100 g/L of trastuzumab were injected in nude mice (n=3). Fluorescence images as well as bright-field images were acquired via a back-thinned CCD-cooled camera ORCAIIBT-512 G (Hamamatsu Photonics Deutschland GmbH, Herrsching am Ammersee, Germany) using a coloured glass long-pass RG 665 filter (Melles Griot, Voisins les Bretonneaux, France). Optical excitation was carried out at 633 nm, and the emission wavelength was detected at 680 nm. Exposure time was set at 30 s for optical imaging and 0.05 s for bright field imaging. Fluorescent images were acquired at different time point to monitor the loss of fluorescence over time resulting in the degradation of the hydrogel.

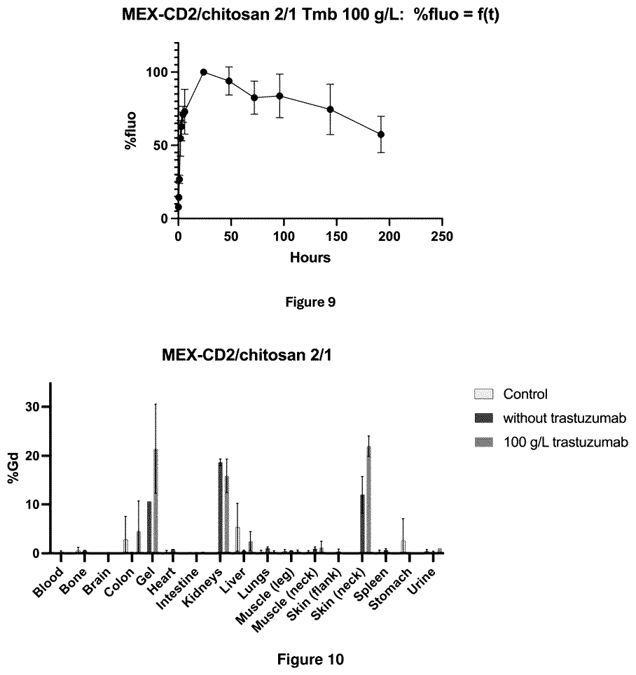

The results are shown on FIG. 8 and FIG. 9.

After 8 days, approximately 55% of the initial fluorescence intensity is observed meaning that a significant degradation of the formed hydrogel occurred during this period.

### 9.2. Study of In Vivo Biodistribution by ICP-MS

Following experiments detailed in the 9.1. section, mouse organs were collected after dissection (21 days after injection) and then mineralized with concentrated nitric acid in a Multiwave 5000 from Anton Paar. Organs were divided and digested according to their weights to ensure homogeneity of digestion throughout the method. Standard 69% HNO₃ [ROTIPURAN Supra provided by Roth] and ultrapure (18.2 MΩcm) water were used for all digestion and sample preparation. Reactors were cleaned before and after sample cycles with the programmed Cleaning Method (4 mL water and 6 mL 69% HNO3, 0 to 180° C. ramping over 10 minutes, 180° C. for 10 minutes). Rinsed with ultrapure water after cycle. Using the Bio-Organic Digestion Method, the samples were digested under the following parameters: 69% HNO3, 0-100° C. ramping over 10 minutes, 100° C. for 10 minutes, 100-200° C. ramping over 10 minutes, and 200° C. for 10 minutes (40-minute cycle).

ICP-MS analyses were carried on a PerkinElmer NexION 2000 Inductively Coupled Plasma Mass Spectrometer (ICP-MS). Before use, the machine was cleaning continuously with 10% HNO3 for 20 minutes then 1% HNO₃ for 1.5 hours. All of the system parameters were optimized before beginning the analysis following the machine's protocols (mass calibration, torch alignment, QID in mode KED and standard, nebulizer gas flow, and dual detector calibration). Once all of the parameters are optimized, a final performance check was done in standard and KED. An internal standard of 2 ppb Indium was used in all ICP samples to ensure a consistent measurement and reliability. The base solvent was composed of 0.5% HNO₃, 0.7% tertiary-butanol, and water. The tertiary-butanol was included to help minimize the matrix effects of carbon, since degradation of the samples included proteins and other carbon-heavy biomolecules. Gadolinium (158Gd) was analyzed.

The results are shown on FIG. 10.

21 days after injection, about 40% of the hydrogel is found in the region of injection and about 20% is in the kidneys which highlights the elimination pathway of the hydrogel after degradation. The hydrogel remaining in the injection area is in the form of an already degraded hydrogel that is slightly adhered to the tissues of the neck.

### 9.3. Study of In Vivo Biodegradability by MRI

Solutions of MEX-CD2-I-HER2 were prepared according to the process of Example 7.2.

MEX-CD2 used for this formulation have some of its pre-grafted chelating agents complexed with gadolinium Gd³⁺ ions. 200 μL of such Gd complexed MEX-CD2-I-HER2 solution was injected into the neck region of 3 mice and 40 μL in 3 other mice to evaluate the degradability profile according to the gel volume administered. 200 μL of a solution without antibody (not according to the invention) have also been injected in 3 mice to evaluate the degradability profile with and without antibody loading. Furthermore, drug-loaded solution has been prepared according to the process explained in Example 7.2. using a 2/4% and a 0/10% viscous solution as the precursor solution respectively before mixing with the antibody solution with a ratio α/β % where α is the mass concentration (w/w) of chitosan A and β the mass concentration (w/w) of total polysaccharide B (comprising unmodified compound and Gd labelled compound). Such solutions have been injected (200 μL) in the neck of 3 mice respectively for each solution.

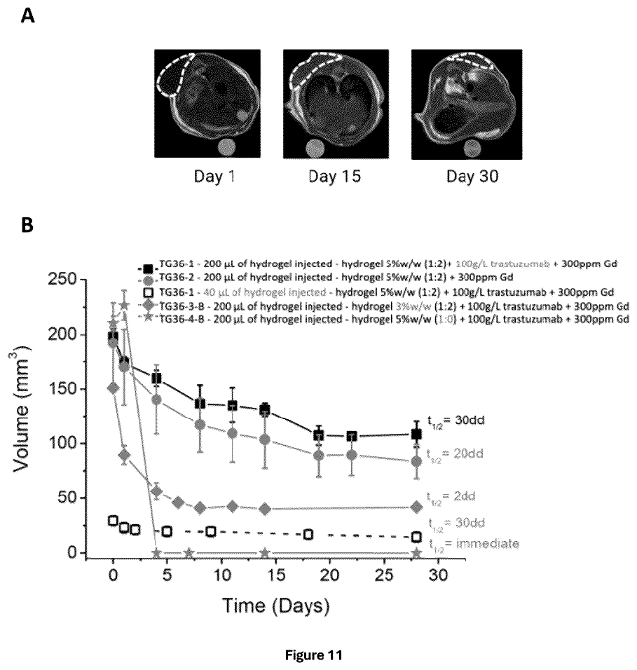

The results are shown on FIG. 11.

Hydrogels formed by MEX-CD2-I-HER2 solution (antibody loaded) display different t_{1/2} values (30 vs. 20 dd) and strongly different T₁ values (ca 500 ms vs. ca. 250 ms, respectively) when compared to hydrogels formed by the reference solution without antibody loading (not according to the invention). The presence of trastuzumab affects both the size decrease and the T₁ change.

Scaffold obtained from the polymeric solution at 3% loaded with antibody has a starting volume lower than the other scaffolds, even if the same volume was injected. This is probably due to the higher water content which is immediately re absorbed upon the administration. It quickly decreases in size (t_{1/2} is ca. 2dd). The decrease of scaffold concentration (from 5% to 3%) affects both the size decrease and the T₁ change.

Scaffold obtained from the polymeric solution solely composed of polysaccharide (i.e. from the 0/10% polymeric precursor solution) show a starting size increase and then a very quick disappearance from the injection site. At t=3 days the scaffold is no longer present. The change of scaffold composition affects both the size decrease and the T₁ change.

Therefore, the hydrogel scaffold obtained from the MEX-CD2-I-HER2 solution appears to be the one with the best formulation. The presence of trastuzumab speeds up the water filling (as detectable in T₁ change, and the not homogenous signal in T_{1w} and T_{2w} MRI).

Furthermore, results obtained on other formulations demonstrate the capacity to tune on demand the hydrogel scaffold formed after injection and hence the release of the protein entrapped (here trastuzumab).

### 9.4. Conclusion Regarding Biodegradability

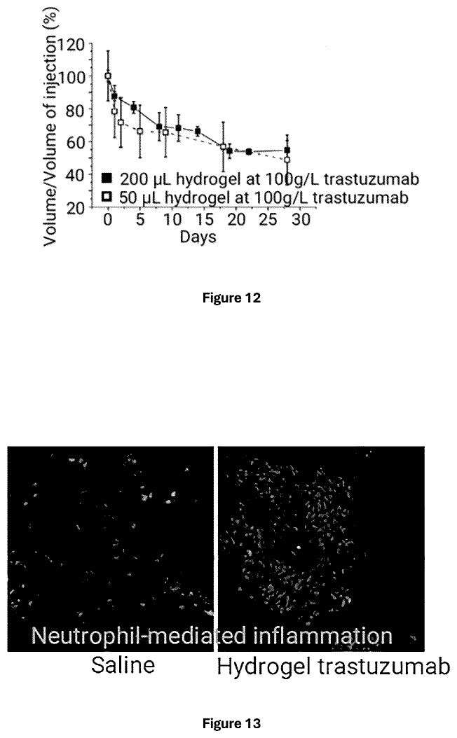

Importantly, once normalized, the volume injected did not interfere with the degradation kinetic of the hydrogel. As such, 50 μL and 200 μL of hydrogel-loaded trastuzumab implanted subcutaneously (n=3) provide the same degradation profile as shown on FIG. 12.

### 9.5. Study of Inflammatory Cells (Neutrophils)

The MEX-CD2-I-HER2 solutions used in this Example were prepared according to the process detailed in Example 7.1.

Ex vivo quantification of IL6, TNFa, and mub-40 levels were assessed by cytometry on digested samples at 24 h and 120 h post-implantation (n=3 per group). Results were compared to negative control (no treatment) and positive control (pre-activated neutrophils with LPS) (data not shown). A slight inflammation was initially observed post-implantation—i.e. increased amounts of pro-inflammatory neutrophils localized at the injection site, increased level of TNFa. However, at day 5 post-implantation, the total amount of pro-inflammatory neutrophils decreased significantly to be back to basal levels as well as for TNFa. The cytokine IL6 was low at day 1 and increased at day 6 (2.5% and 13%). However, these levels are significantly lower than for positively activated neutrophils (54%), confirming the biocompatibility of the hydrogel formulation.

The absence of pro-inflammatory neutrophils at the implantation site was further validated by immunofluorescence staining with an anti-mub40 antibody (day 5 post-implantation, n=3).

The results are shown on FIG. 13.

### 9.6. Conclusions Regarding Safety Profile of the Hydrogel-Loaded Monoclonal Antibody Formulation

The results show that the presence of the gel under the skin does not induce inflammation/much less inflammation that gels of the prior art such as gels comprising, among others, alginate.

### Example 10: In Vitro Study of the Release of Various Proteins by the Hydrogel

### 10.1. Material and Methods

The MEX-CD2-I solutions used in this Example were prepared according to Example 7.1 except that the solution is loaded with trastuzumab (Roche), rituximab (Roche), trastuzumab, HER2 Fab′2, HER2 VHH. The mix of the solution was prepared overnight before to place the samples in a 1 mL Eppendorf tube filled with PBS. At each time point, an aliquot was harvested from the milieu and fresh PBS was added to the solution. A Bradford assay was performed to quantify the amount of proteins in each aliquots to quantify the amount of antibodies/fragments/proteins.

### 10.2. Results

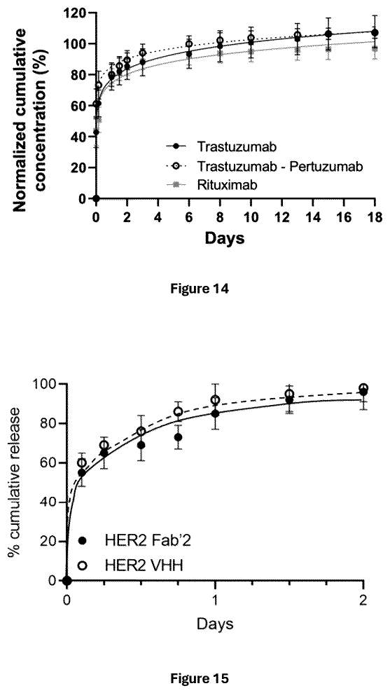

The results regarding the cumulative release of monoclonal antibodies alone (trastuzumab or rituximab) and combination of monoclonal antibodies (trastuzumab and pertuzumab) in PBS at 37° Celsius are shown on FIG. 14.

The results regarding the cumulative release of fragment of monoclonal antibodies (Fab′2 and VHH) in PBS at 37° Celsius are shown on FIG. 15.

### Example 11: SEM Imaging of a Formulation MEX-CD2-I-HER2 in Accordance with the Invention

Solutions of MEX-CD2-I-HER2 were prepared according to the process of Example 7.2.

MEX-CD2 used for this formulation have some of its pre-grafted chelating agents complexed with gadolinium Gd³⁺ ions. 500 μL of such Gd complexed MEX-CD2-I-HER2 solution was injected in 100 mL of a 10 mmol/L PBS solution (Phosphate Buffered Saline). 500 μL of a solution without antibody (not according to the invention) have also been injected in 100 mL of a 10 mmol/L PBS solution to further evaluate the impact of antibody loading on the surface morphology of the formed hydrogel by SEM imaging. Both solutions were injected with a Terumo agani 25 G×16 mm needle with an internal diameter of 284 μm measured by optical microscopy. After 1 h of gelation, PBS is replaced by 100 mL of a 10% (V/V) EtOH/H₂O solution. Every hour the solution is replaced by another solution whose ratio increase by 20% (V/V) in ethanol up to 100% EtOH. The samples then undergo critical point drying (CPD) comprising 12 cycles of 120 s at 18° C. to change from 100% EtOH to gaseous CO₂. Resulting dehydrated hydrogel are then placed on a metal STUB and are metallized by depositing a thin layer of 10 nm of copper on the surface of the polymeric scaffold. Obtained scaffolds are observed with a SEM FEI QUANTA 250 FEG at 2 kV or 3 kV.

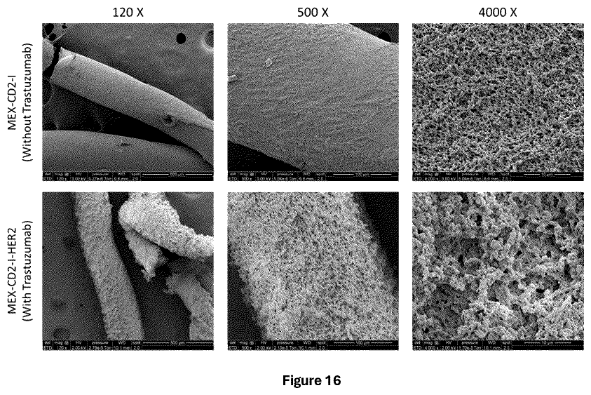

The results are shown in FIG. 16.

The diameter of the gel measured in both cases is slightly lower (average of 260 μm) than the internal diameter of the needle used for the injection (284 μm). Hydrogel scaffold obtained with the solution loaded with trastuzumab highlight a network significantly thicker compared to the scaffold obtained from the solution without antibody. A coarser network is obtained which is resulting from the gelation of the system in presence of a highly antibody concentrated medium. This difference in the surface morphology demonstrates the capacity of the gel to entrap the drug and release it after gelation.

1-15. (canceled)

16. A pharmaceutical formulation of a pharmaceutically active protein comprising:
an effective amount of a protein or a combination of proteins,
a chitosan A comprising between 90 and 100 mol % of D-glucosamine and between 0 and 15% of N-acetyl-D-glucosamine,
at least one statistic polysaccharide B comprising D-glucosamine, N-acetyl-D-glucosamine and at least one saccharide unit of formula I: 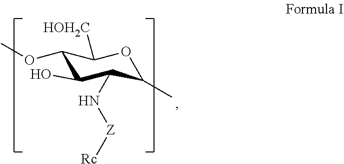
wherein:
Rc is a hydrophilic group,
Z is a linker being a single bond, a hydrocarbonated chain comprising between 1 and 12 carbon atoms, said chain being linear or branched, saturated or unsaturated and optionally comprising one or more heteroatoms, and
one or more pharmaceutically acceptable excipients.

17. The pharmaceutical formulation according to claim 16, wherein Rc is a group having acidic properties, typically selected from the group comprising carboxyl group (—COOH), sulfonic group (—SO₂OH), phosphonate groups (—PO(OH)₂), thiol group (—SH), alcohol group (—OH) and groups comprising a chelating agent.

18. The pharmaceutical formulation according to claim 16, wherein said statistic polysaccharide B is of the general formula II: 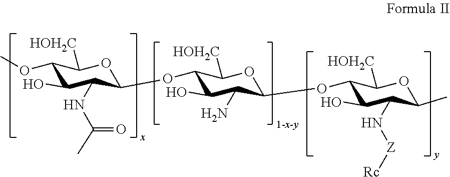
wherein:
Rc is a hydrophilic group,
Z is a linker being a single bond, a hydrocarbonated chain comprising between 1 and 12 carbon atoms, said chain being linear or branched, saturated or unsaturated and optionally comprising one or more heteroatoms,
x is comprised between 0.01 and 0.5,
y is comprised between 0.05 and 0.5,
a ratio y/x is greater than 0.2, preferably greater than 1,
a sum x+y is greater than 0.1.

19. The pharmaceutical formulation according to claim 16, wherein the viscosity of the formulation is comprised between comprised between 10 and 500 Pa·s, as measured at room temperature by rotational rheometry in plane cone geometry at a shear rate of between 0.001 s⁻¹ and 0.01 s⁻¹.

20. The pharmaceutical formulation according to claim 16, wherein the pH of the formulation is comprised between 5.0 and 6.5.

21. The pharmaceutical formulation according to claim 16, wherein the osmolarity of the formulation is comprised between 50 and 600 mOsm/L.

22. The pharmaceutical formulation according to claim 16, comprising:
between 5 and 200 g/l of chitosan A and between 10 and 200 g/l of statistic polysaccharide B, and
between 1 g/l and 200 g/l of protein or combination of proteins.

23. The pharmaceutical formulation according to claim 16, wherein the protein has a molecular weight comprised between 10 kDa and 250 kDa.

24. The pharmaceutical formulation according to claim 16, wherein the protein or combination of proteins is selected from the group consisting of an antibody, an enzyme, a fusion protein, and a combination thereof.

25. The pharmaceutical formulation according to claim 16, wherein the protein or combination of proteins includes an antibody or an antigen-binding fragment of an antibody.

26. The pharmaceutical formulation according to claim 25, wherein the antibody is selected from the group consisting of trastuzumab, rituximab and pertuzumab or combinations thereof.

27. The pharmaceutical formulation according to claim 16, further comprising at least one pharmaceutically acceptable excipient selected from solvents, stabilizers, surfactants, buffering agents, antimicrobial preservatives, protectants, antioxidants, chelating agents, and bulking agents.

28. The pharmaceutical formulation according to claim 16, wherein the formulation comprises:
between 1 and 200 g/l of an antibody or a combination of antibodies,
between 5 and 200 g/l of chitosan A, and
20 and 400 g/l of statistic polysaccharide B, and at least one excipient selected from:
between 1 and 100 mM of a buffering agent providing a pH between 5.5 and 6.5;
between 1 and 500 mM of a stabilizer; and
between 5 and 25 mM of an antioxidant.

29. A kit comprising:
at least one first container containing the chitosan A and the statistic polysaccharide B as defined in the pharmaceutical formulation according to claim 16, and
at least one second container containing a protein or a combination of proteins as defined in said pharmaceutical formulation, wherein said protein has a molecular weight comprised between 10 kDa and 250 kDa or said combination of proteins have molecular weights comprised between 10 kDa and 250 kDa.

30. A method for treatment of cancer comprising administering a subject in need thereof the pharmaceutical formulation according to claim 16 or a kit comprising:
at least one first container containing the chitosan A and the statistic polysaccharide B as defined in said pharmaceutical formulation, and
at least one second container containing a protein or a combination of proteins as defined in said pharmaceutical formulation, wherein said protein has a molecular weight comprised between 10 kDa and 250 kDa or said combination of proteins have molecular weights comprised between 10 kDa and 250 kDa.

31. The pharmaceutical composition according to claim 16, wherein the heteroatom is selected from nitrogen, oxygen and sulfur.

32. The pharmaceutical formulation according to claim 18, wherein the protein has a molecular weight comprised between 100 kDa and 250 kDa.

33. The pharmaceutical formulation according to claim 18, comprising between 50 and 100 g/l of protein or combination of proteins.

34. The pharmaceutical composition according to claim 28, wherein the pharmaceutical composition comprises an antibody selected from selected from the group consisting of trastuzumab, rituximab, pertuzumab and daratumumab or a combination of trastuzumab and pertuzumab.

35. The method according to claim 30, wherein the cancer is a breast cancer or a gastric cancer.
